(19) United States
(12) Patent Application Publication
(10) Pub. No.: US 2026/0314923 A1
(43) Pub. Date: Oct. 15, 2026

(54) OPHTHALMOLOGIC APPARATUS

(71) Applicant: NIDEK CO.,LTD., Gamagori-shi (JP)

(72) Inventors: Ai TAKAYA, Gamagori-shi (JP); Norimasa SATAKE, Gamagori-shi (JP); Junpei NISHIYAMA, Gamagori-shi (JP); Shinya IWATA, Gamagori-shi (JP); Moe KONDO, Gamagori-shi (JP); Junji TAMARAI, Gamagori-shi (JP)

(21) Appl. No.: 19/461,705

(22) Filed: Jan. 27, 2026

Related U.S. Application Data
(63) Continuation of application No. PCT/JP2024/027682, filed on Aug. 2, 2024.

(30) Foreign Application Priority Data
Aug. 3, 2023 (JP) 2023-127346
Jan. 9, 2024 (JP) 2024-001539
Mar. 29, 2024 (JP) 2024-057472

(51) Int. Cl. A61B 3/00

(52) U.S. Cl. CPC A61B 3/0083; A61B 3/0025

(57) ABSTRACT

An ophthalmic apparatus includes: a face support unit configured to support a subject's face; an examination unit configured to examine a subject's eye; a drive unit configured to move the examination unit three-dimensionally; an imaging unit configured to acquire an alignment image including at least one of left and right subject's eyes; and a controller configured to display the alignment image. The controller is further configured to display alignment assistance information indicating whether a position of the subject's eye is within (i) a first range that is a movable range of the examination unit on the alignment image by the drive unit or (ii) a second range on the alignment image that is outside the first range.

### CROSS REFERENCE TO RELATED APPLICATION

This application is a continuation application of International Patent Application No. PCT/JP2024/027682 filed on Aug. 2, 2024, which designated the U.S. and claims the benefit of priority from Japanese Patent Application No. 2023-127346 filed on Aug. 3, 2023, Japanese Patent Application No. 2024-001539 filed on Jan. 9, 2024, and Japanese Patent Application No. 2024-057472 filed on Mar. 29, 2024. The entire disclosure of all of the above applications is incorporated herein by reference.

### TECHNICAL FIELD

The present disclosure relates to an ophthalmologic apparatus.

### BACKGROUND

Various ophthalmologic apparatuses for examining a subject's eye are known. For example, examples of the ophthalmologic apparatus include an eye refractive power measurement apparatus, a wavefront aberration measurement apparatus, a tonometer, an optical coherence tomography (Optical Coherence Tomography: OCT) apparatus, a fundus camera, a perimeter, a corneal topography measurement apparatus, a corneal endothelial imaging apparatus, and the like. These ophthalmologic apparatuses perform an examination in a state where an examination unit is aligned (positioned) at an appropriate position with respect to the subject's eye.

As one alignment method for an ophthalmologic apparatus, a method has been proposed in which the position of an examination unit is moved in accordance with the position of the subject's eye in a wide-range alignment image obtained by capturing an image of a subject's face or the like (see Patent Document 1 (JP 2017-217120 A)).

In addition, many stationary ophthalmologic apparatuses include a face support unit that supports a face. Patent Document 1 discloses a face support unit including a mechanism for adjusting the height of a chin rest on which a subject's chin is placed in order to adjust the height of the subject's line of sight.

### SUMMARY

For example, it is conceivable that a movable range of an inspection unit in the vertical direction is narrower than an angle of view of an alignment image. When an eye to be inspected is positioned outside the movable range of the inspection unit, alignment cannot be appropriately performed only by moving the inspection unit. In this case, it is necessary to adjust a height of a subject's line of sight by, for example, adjusting a height of a chin rest. However, the examiner was unable to ascertain whether it is necessary to adjust the height of the chin rest. For this reason, regardless of the position of the eye to be inspected in an initial state, there are cases where the time required for the inspection is prolonged because the height adjustment of the chin rest is inevitably performed for each inspection. Further, when alignment is first attempted by moving only the inspection unit without adjusting the height of the chin rest, if the eye to be inspected is positioned outside the movable range of the inspection unit, it becomes necessary to adjust the chin rest at a stage when the inspection unit has reached its movement limit and to move the inspection unit again. Since the inspection unit moves twice, the time required for the inspection is also prolonged in this case.

The present disclosure has been made based on at least one of the problems of the conventional technology, and an object thereof is to provide an ophthalmic apparatus that is likely to improve examination efficiency.

An ophthalmic apparatus according to a first aspect of the present disclosure includes: a face support unit configured to support a subject's face; an examination unit configured to examine a subject's eye; a drive unit configured to move the examination unit three-dimensionally; an imaging unit configured to acquire an alignment image including at least one of left and right subject's eyes; and a controller configured to display the alignment image. The controller is further configured to display alignment assistance information indicating whether a position of the subject's eye is within (i) a first range that is a movable range of the examination unit on the alignment image by the drive unit or (ii) a second range on the alignment image that is outside the first range.

A method according to a second aspect of the present disclosure includes: supporting, with a face support unit, a subject's face; examining, with an examination unit, a subject's eye; moving, with a drive unit, the examination unit three-dimensionally; acquiring, with an imaging unit, an alignment image including at least one of left and right subject's eyes; and displaying, with a controller, the alignment image. The method further comprises displaying alignment assistance information indicating whether a position of the subject's eye is within (i) a first range that is a movable range of the examination unit on the alignment image by the drive unit or (ii) a second range on the alignment image that is outside the first range.

According to the present disclosure, examination efficiency is likely to be improved.

### BRIEF DESCRIPTION OF THE DRAWINGS

FIG. 1 is a diagram illustrating a schematic configuration of an ophthalmic imaging apparatus according to one embodiment.

FIG. 2 is a diagram illustrating an overview of an optical system of the apparatus according to one embodiment.

FIG. 3 is a flowchart illustrating a flow of operations of the apparatus according to one embodiment.

FIG. 4 is a selection screen in the embodiment, and is an example of display in an OCT/FC mode.

FIG. 5 is a selection screen in the embodiment, and is an example of display in an anterior-segment OCT mode.

FIG. 6 is a selection screen according to one embodiment and is an example of display in an anterior-segment OCT mode.

FIG. 7 is an alignment screen according to one embodiment where a face image is displayed as an observation image.

FIG. 8A is an example of display of a face image when detection of the subject's eye has succeeded.

FIG. 8B is an example of display of a face image when detection of the subject's eye has failed.

FIG. 9 is a diagram illustrating a relationship between a region on a face image and a movable range of an imaging unit.

FIG. 10 is an alignment screen according to one embodiment where an anterior-segment image is displayed as an observation image.

FIG. 11 is an observation screen according to one embodiment where a macula multi is selected as a scan pattern.

FIG. 12 is a confirmation screen corresponding to FIG. 11.

FIG. 13 is an observation screen according to one embodiment where a macula map is selected as a scan pattern.

FIG. 14 is a confirmation screen corresponding to FIG. 13.

FIG. 15 is an observation screen according to one embodiment where FC imaging is selected.

FIG. 16 is a confirmation screen corresponding to FIG. 15.

FIG. 17 is an observation screen according to one embodiment where a macula line is selected as a scan pattern.

FIG. 18 is a confirmation screen corresponding to FIG. 17.

FIG. 19 is a report output screen according to one embodiment.

FIG. 20 illustrates an enlarged report.

FIG. 21 is a setting screen according to one embodiment.

FIG. 22 is a setting screen according to one embodiment.

FIG. 23 is a confirmation screen according to one modification example, in which a wide-area map (retinal map) including the macula and the optic disc is selected as a scan pattern.

FIG. 24 is a report output screen according to one modification example.

### DESCRIPTION OF THE EMBODIMENT

Embodiments according to the present disclosure will be described. Each embodiment can be applied to some or all of other embodiments. For example, the following “<>” the items classified in “<>” can be used independently or in association with each other.

### 1. First Embodiment

The ophthalmic apparatus according to the first embodiment examines eye characteristics of a subject's eye. The ophthalmic apparatus may be a stationary apparatus. In this case, the ophthalmic apparatus examines the subject's eye while being installed on a table or the like.

In the first embodiment, the ophthalmic apparatus includes at least a face support unit, an examination unit, a drive unit, an imaging unit, and a controller. The ophthalmic apparatus may additionally include a display unit.

The face support unit supports a subject's face. The face support unit may include a chin rest and a chin-rest height adjustment mechanism. The chin rest is for placement of the subject's chin. The chin-rest height adjustment mechanism is a mechanism for changing the height of the chin rest. Unless otherwise noted, in the first embodiment, the chin-rest height adjustment mechanism will be described as having a driver (actuator) that changes the height of the chin rest based on a control signal. However, the present disclosure is not necessarily limited thereto, and a mechanical mechanism may be used.

The examination unit is used to examine the subject's eye. The examination unit may include, for example, at least one of various examination optical systems such as an ocular refractive power measurement optical system, a corneal curvature measurement optical system, an intraocular pressure measurement optical system, a fundus imaging optical system, and an OCT optical system. In the examination unit, an examination window for acquiring eye characteristics of the subject's eye is disposed. The examination is performed in a state where the subject's eye faces the examination window with a predetermined distance therebetween.

The drive unit moves the examination unit three-dimensionally. For an examination, the positional relationship between the subject's eye and the examination unit is adjusted in advance. With respect to the XY directions, the positional relationship is adjusted such that an examination axis passing through the examination window coincides with a predetermined position of the subject's eye (e.g., the pupil center, the corneal apex, etc.). However, the movable range of the examination unit by the drive unit has mechanical limitations. Therefore, the subject's eye needs to be positioned within a range in which the examination axis can reach by moving the examination unit.

The imaging unit acquires an alignment image including at least one of the left and right subject's eyes. The imaging unit may acquire, as the alignment image, a facial image including both the left and right subject's eyes. Alternatively, the imaging unit may acquire an anterior-segment image as the alignment image. The anterior-segment image may be an image captured from the front of the anterior segment, or may be an image captured from an oblique direction. The imaging unit may be disposed in the examination unit. An optical system used by the imaging unit to acquire the alignment image (alignment optical system) may share at least a part with an examination optical system for measuring eye characteristics, or may be provided separately.

The controller of the first embodiment sets a first range and a second range for the alignment image. The first range indicates a movable range of the examination unit by the drive unit. In other words, the first range indicates a range in which the examination axis can reach by moving the examination unit. However, the first range does not need to completely coincide with the movable range of the examination unit, and may substantially coincide therewith. The second range indicates a region outside the first range. In other words, the second range indicates a range in which the examination axis cannot reach even by moving the examination unit. In the first embodiment, at least the second range is set in the vertical direction of the first range. However, the present disclosure is not necessarily limited thereto, and the second range may be set in the left-right direction of the first range.

Further, the controller of the first embodiment may display an alignment image.

The controller of the first embodiment may display alignment assistance information indicating which of the first range and the second range on the alignment image the position of the subject's eye is within. For example, when an alignment image including both the first range and the second range is displayed, the controller may, as the alignment assistance information, visually indicate, on the alignment image, a boundary between the first range and the second range. For example, such a boundary line may be superimposed and displayed on the alignment image. Alternatively, the boundary may be visually indicated by processing the second range in the alignment image. For example, a mask may be superimposed on the second range, or contrast, brightness, gamma, and the like in the second range may be corrected, or blurring, mosaic processing, or the like may be performed on the second range. In this manner, the controller may display an image obtained by performing image processing that reduces visibility in the second range as compared with the first range.

Further, for example, the controller may display, as the alignment assistance information, information indicating whether or not the subject's eye is positioned on an image on which the subject's eye is displayed, separately from the image. For example, the controller may detect the subject's eye in at least one of the first range and the second range of the alignment image, and may display the alignment assistance information depending on whether or not the subject's eye is present in the detection range. The alignment assistance information to be displayed may be text information, or may be information in other forms. Such display is particularly effective, for example, when, of the first range and the second range, the first range is extracted (trimmed) and displayed on the screen.

In the first embodiment, whether or not the position of the subject's face should be adjusted can be appropriately grasped by the examiner based on the position of the subject's eye on the alignment image and the alignment assistance information. Accordingly, the time required for alignment tends to be shortened, and thus examination efficiency tends to be improved.

The controller sets the position of the subject's eye on the alignment image based on various methods. For example, the controller may detect the subject's eye from the alignment image, and the position of the subject's eye may be set based on a detection result of the subject's eye. Further, the controller may set the position of the subject's eye to a specified position by receiving, via a user interface, an operation input specifying the position of the subject's eye on the alignment image. In the alignment image, a region (ROI) in which the position of the subject's eye can be set may be appropriately set.

When the controller determines that the subject's eye is positioned in the first range on the alignment image, the controller may move the examination unit in accordance with the position of the subject's eye and may control the examination unit to perform an examination. In determining whether the subject's eye is positioned within the first range, the presence or absence of the subject's eye in the first range may be detected based on the alignment image. When an operation input for specifying the position of the subject's eye is performed with respect to any position in the first range, it may be determined that the subject's eye is positioned at the specified position.

Further, for example, when the controller determines that the subject's eye is positioned within the second range on the alignment image, the controller may execute guidance processing for guiding movement of the subject's face. In determining whether the subject's eye is positioned within the second range, the presence or absence of the subject's eye in the second range may be detected based on the alignment image. Further, a case where the subject's eye is not detected in the first range may be regarded and determined as a case where the subject's eye is positioned in the second range.

In the guidance processing, the controller may control a chin-rest height adjustment mechanism to adjust a height of the chin rest in accordance with a position of the subject's eye on the alignment image. Accordingly, even when the subject's eye is detected in the second range, the subject's eye is automatically placed in the first region on the alignment image. Further, in the guidance processing, the controller may output an operation guide for adjusting the height of the chin rest. The operation guide may be a visual guide output from a display unit. Further, the operation guide may be an audio guide output from a speaker.

Further, when it is determined that the position of the subject's eye on the alignment image is within the first range, an operation input for advancing the examination may be enabled, and when it is determined that the position of the subject's eye on the alignment image is within the second range, the operation input for advancing the examination may be disabled. Between a case where it is determined that the position of the subject's eye on the alignment image is within the first range and a case where it is determined that the position of the subject's eye on the alignment image is within the second range, a display mode of a GUI widget used for the operation input for advancing the examination, or other graphics, may be switched.

Further, when the subject's eye is not detected in the alignment image, the controller may output error information indicating a detection error. In a case of a detection error or false detection, the position of the subject's eye on the alignment image may be correctable. The controller may correct the position of the subject's eye based on an operation input via the alignment image. For example, when the position of the subject's eye on the alignment image is specified via a user interface, the specified position, or a position of the subject's eye detected in a region near the specified position, may be used as a corrected position.

### 2. Second Embodiment

### Background Art and Technical Problem in Second Embodiment

In recent years, in the ophthalmic field, apparatuses capable of capturing an ophthalmic image of a subject by a plurality of types of imaging operations have been increasingly becoming widespread. As an example, an OCT apparatus may be mentioned. In the OCT apparatus, a plurality of scan patterns are combined to capture, as an ophthalmic image, an OCT image of the subject's eye based on each of the scan patterns (see Patent Document 2 (JP 2017-153826 A)).

In some cases, a report based on the captured ophthalmic image is generated by the apparatus that performed the imaging. In the report, one or more of at least either an ophthalmic image and an analysis result for the ophthalmic image is/are arranged. At the facility, the generated report is stored after being confirmed by an examiner. (2) When a plurality of imaging operations are performed on one subject, it is conceivable that a plurality of types of reports are generated.

The second embodiment of the present disclosure has been made based on at least one of the problems of the conventional art described in (2), and has a technical object to provide an ophthalmic apparatus that can suitably confirm a report generated based on a plurality of imaging operations.

### Details of Second Embodiment

An ophthalmic apparatus according to the second embodiment includes at least an imaging unit, a first selection unit, a second selection unit, an imaging control unit, and a display control unit.

The imaging unit is used to image a subject's eye. In the second embodiment, the imaging unit is capable of capturing an ophthalmic image of a subject by a plurality of types of imaging operations. For example, the imaging unit may include at least one of various imaging optical systems, such as a fundus imaging optical system and an OCT optical system. The ophthalmic image may be an image of either a fundus region or an anterior segment region. The ophthalmic image may be at least one of a front image, a fluorescence image, an intensity OCT image (hereinafter, simply referred to as an “OCT image”), and an OCT motion-contrast image (hereinafter, simply referred to as a “motion-contrast image”).

A first selection unit accepts a first selection operation for selecting a subject. The first selection operation may be an operation of selecting, via the first selection unit, any one of identification information (ID, name, age, etc.) of a plurality of subjects registered in advance in a database. When the subject is not registered in the database, the first selection operation may be an operation of newly registering (inputting) identification information of the subject in the database and selecting the subject. For example, the identification information is input by reading, with a barcode reader connected to the apparatus, a barcode describing the subject's identification information, or by direct input via a keyboard. When imaging a subject whose identification information has already been registered, the identification information may be retrieved by displaying a subject list and selecting any corresponding one from the list.

A second selection unit accepts a second selection operation. The second selection operation is an operation for selecting, as an examination pattern indicating examination contents for the selected subject, an examination pattern including two or more types of imaging operations from among a plurality of examination patterns prepared in advance.

When an examination pattern including two or more types of imaging operations is selected and the examination is started, the imaging control unit sequentially captures ophthalmic images of a subject in each imaging operation.

An examination pattern including two or more types of imaging operations is prepared to successively perform a plurality of imaging operations on one subject's eye. The examination pattern may be a pattern in which a plurality of scan patterns by OCT are combined. Each scan pattern specifies a position on tissue at which an OCT image is acquired and a number of OCT images to be acquired. Examples of scan pattern types include a line scan, a cross scan, a multi scan, a radial scan, and a raster scan (also referred to as a map scan). For these scan patterns, scan patterns whose positions on tissue and scan lengths differ from each other may be prepared. Also, for example, as an examination pattern including two or more types of imaging operations, there may be a pattern in which a plurality of imaging operations having different image types are combined. For example, the pattern may be a pattern for successively capturing an OCT image and a front fundus image, or may be a pattern for successively capturing an OCT image and a motion contrast image.

The display control unit generates a plurality of reports based on a plurality of ophthalmic images acquired by the respective imaging operations for a subject. Depending on the examination pattern, one or a plurality of reports may be generated, or a report may be generated for each imaging operation included in the examination pattern.

Furthermore, the display control unit displays a list of a plurality of reports on a report list screen. In each report, one or more of at least an ophthalmic image and an analysis result for the ophthalmic image is arranged. The layout of each report may be predetermined. Further, in each report, any of subject identification information, an examination date and time, and an imaged eye (an eye to be imaged, of a left eye or a right eye), etc. may be described in the report.

Furthermore, on the report list screen, information indicating at least any of a type of an imaging operation, an examination date and time, and an imaged eye, with respect to the imaging operation on which the report is based, may be arranged around each report. These pieces of information arranged around the report may overlap those described in the report. On the report list screen, since the information described in the report is reduced in size and thus readability is likely to be impaired, it is useful that information indicating an outline of the report is displayed larger around the report.

On the report list screen, UI elements (GUI widgets) for receiving operations by an examiner may be arranged. For example, the UI elements are operated such that the examiner selects one or more desired reports from among a plurality of reports arranged on the report list screen and saves the selected report(s) in a storage medium and/or transfers the selected report(s) to an external device. On the report list screen, any desired number of reports may be selectable collectively at one time.

As a result of the display as described above, the examiner can suitably check reports generated based on a plurality of imaging operations.

The display control unit may cause the report list screen to transition from the confirmation screen. The confirmation screen is a screen displayed immediately after imaging and is used to check an imaging result. For example, on the confirmation screen, at least an ophthalmic image obtained by the immediately preceding imaging may be displayed. In addition, any of an observation image, various indices (image-quality information of a captured image or an observation image), and information indicating an alignment state, for allowing the examiner to determine success or failure of imaging, may be displayed. In addition, UI elements for receiving operations by the examiner may be arranged. On the confirmation screen, at least any of a UI element operated to perform re-imaging of an ophthalmic image and a UI element operated to finalize success or failure of imaging may be arranged.

The display control unit may cause the report list screen to transition from the observation screen. In this case, the confirmation screen is omitted. In this case, as UI elements, elements that receive an operation for executing at least one of re-imaging of an ophthalmic image and finalizing success or failure of imaging may be arranged on the report list screen. By operating the UI element for finalizing success or failure of imaging, a report based on the ophthalmic image displayed on the confirmation screen may be generated, the ophthalmic image may be stored in a memory, or the ophthalmic image may be transferred to an external destination. After selecting one or more reports displayed on the list screen, by further operating any of these UI elements, re-imaging of the ophthalmic image serving as a basis of the report is performed or success or failure is finalized.

A report displayed on the report list screen may be a thumbnail image of the report. By selecting the thumbnail image, the report image may be displayed in an enlarged manner.

When an examination pattern for capturing each of the right eye and the left eye is selected and ophthalmic images of the right eye and the left eye are captured, a right-eye display area and a left-eye display area may be preset on the report list screen. Further, in this case, reports based on ophthalmic images of the right eye are displayed in a list in the right-eye display area, and reports based on ophthalmic images of the left eye are displayed in a list in the left-eye display area. Accordingly, an examiner can intuitively recognize the reports corresponding to the right eye and the left eye, respectively.

Further, in this case, the reports displayed in the right-eye display area and the reports displayed in the left-eye display area may be arranged at mutually corresponding positions in accordance with at least one of the type of imaging operation and the imaging date of the imaging operation on which each report is based. For example, when the same imaging operation is performed for each of the right eye and the left eye and reports based on the same imaging operation are generated for each of the right eye and the left eye, those right-eye and left-eye reports may be arranged at corresponding positions between the right-eye display area and the left-eye display area. Accordingly, an examiner can intuitively recognize whether ophthalmic images corresponding to each of the right eye and the left eye have been captured without omission. In addition, reports based on imaging performed on the same imaging date for each of the right and left eyes may be arranged at corresponding positions in the right-eye display area.

### 3. Third Embodiment

### Background Art and Technical Problems in Third Embodiment

Various ophthalmic apparatuses for examining a subject's eye are known. For example, conventionally, examples of ophthalmic apparatuses include an eye refractive power measurement apparatus, a wavefront aberration measurement apparatus, a tonometer, an optical coherence tomography (Optical Coherence Tomography: OCT) apparatus, a fundus imaging apparatus, a perimeter, a corneal shape measurement layer, a corneal endothelium imaging apparatus, and the like. Among these ophthalmic apparatuses, Optical Coherence Tomography (OCT), a fundus camera, an SLO, a perimeter, and the like, each of which performs an examination in a state where an examination unit is aligned (positioned) at an appropriate position with respect to the subject's eye, are widely used in medical institutions and examination facilities (hereinafter collectively referred to as “facilities, etc.”).

As one of alignment methods for an ophthalmic apparatus, there is a method of moving the position of the examination unit in accordance with the position of the subject's eye in a wide-range alignment image obtained by imaging the subject's face or the like, and, in OCT, a tomographic image of the subject's eye is acquired as an OCT image by controlling an OCT optical system and scanning measurement light with respect to an acquisition position of the subject's eye. In OCT, any one of various scan patterns such as a line scan, a cross scan, a multi-scan, a radial scan, and a raster scan is selected for each examination. Among these scan patterns, it is possible to select a combo pattern in which two or more scan patterns are successively executed in a predetermined order. A series of imaging operations based on the combo pattern is hereinafter referred to as combo imaging.

In addition, in OCT for imaging the fundus, it has been proposed to acquire the axial length of the subject's eye and to correct the scan length and/or to use the axial length in analysis processing. Furthermore, it has also been proposed to acquire the axial length of the subject's eye in OCT (see Patent Document 3 (JP 2023-018548 A)).

In addition, many devices in which two or more ophthalmic modalities, such as a stationary optical coherence tomography (Optical Coherence Tomography: OCT) apparatus, a fundus camera, an SLO, and a perimeter, are integrated are also known to include a face support unit that supports the face. Patent Document 4 (JP 2022-029174A) discloses a device integrated with a face support unit and including a mechanism for adjusting the height of a chin rest on which an OCT and a perimeter are placed, in order to adjust the height of the subject's line of sight. (3) It has been found that, when an axial length is acquired in combo imaging, if the axial-length acquisition operation is skipped, the following inconveniences may occur. For example, depending on device operation at a facility, there may be cases where there is no opportunity to reflect the axial length in imaging results obtained by the respective scan patterns captured in combo imaging, at timings other than when imaging is performed. In this case, the accuracy of measurement and analysis for each examination becomes unstable. Further, for example, when analysis based on each scan pattern is performed immediately after imaging, if the axial length is not measured in advance, appropriate measurement and analysis results cannot be obtained. Similar problems may also arise in a device in which two or more modalities are integrated, when an examination result of one modality is used for correction and analysis of measurement results or examination results of another modality.

In response thereto, a third embodiment of the present disclosure has been made based on at least one of the problems of the conventional art described in (3), and has a technical object of providing an ophthalmic apparatus that appropriately performs a combo examination.

### Details of Third Embodiment

An ophthalmic apparatus according to a third embodiment includes at least an examination unit and a control unit. The eye examination apparatus may additionally include an analysis processing unit.

The examination unit performs a plurality of types of examinations on a subject's eye. The examination unit may include, for example, an optical system used for examining the subject's eye. The control unit controls the examination unit. The examination unit may be, for example, at least a fundus OCT. The examination unit may include two or more modalities among a fundus OCT, a fundus frontal image capturing device (a fundus camera, an SLO, etc.), and a perimeter, etc.

The control unit is a processor that performs various types of control in the ophthalmic apparatus (for example, imaging/measurement control and display control).

In the third embodiment, the control unit executes a combo examination. Here, an examination in which two or more of a plurality of types of examinations are successively executed in a predetermined order is referred to as a combo examination. In the combo examination in the third embodiment, a plurality of types of examinations may be successively performed on at least one of the left and right subject's eyes.

The control unit is capable of accepting an operation input for skipping any examination whose turn has arrived, during the combo examination. By skipping an examination, the process transitions to the next examination in the order without performing the skipped examination, or, if there is no next examination, the entire combo examination ends. Further, in the third embodiment, skipping based on an operation input is restricted in a combo examination for a specific examination that is one of the plurality of types of examinations. The specific examination may affect other examination results, among the examinations included in the combo examination, in outputting an examination result (a measurement result and an analysis result) for the combo examination. In this case, since the result of the specific examination may be reflected in the examination result for the combo examination, the accuracy of measurement and analysis for each examination is more likely to be stabilized regardless of apparatus operation at a facility.

In this case, it is desirable that the control unit be configured such that the execution order of each examination in the combo examination is set so that the order of the specific examination comes before that of the other examinations. For example, even when analysis by each examination is performed immediately after the examination, since the examination result of the specific examination is acquired in advance, appropriate examination results can also be obtained for the remaining examinations.

### In the Case of OCT

Here, a description will be given of a case where the examination unit includes an OCT optical system capable of scanning tissue of a subject's eye with a plurality of scan patterns. The OCT optical system is capable of scanning tissue of the subject's eye with a plurality of scan patterns. The OCT optical system may irradiate the subject's eye with measurement light and detect a spectral interference signal between the measurement light and reference light. The OCT optical system includes an optical scanner for scanning the measurement light in a plurality of scan patterns.

In this case, the control unit, as a combo examination, successively executes two or more of a plurality of scan patterns in a predetermined order. Each scan pattern may differ from one another in an acquisition position and range of OCT data. The control unit is capable of accepting an operation input for skipping any scan pattern whose execution order has come during the combo examination. In the combo examination, skipping of a specific scan pattern, which is one of a plurality of types of examinations, based on an operation input is restricted.

Here, the control unit acquires axial length information of the subject's eye as an examination result of the subject's eye obtained by the specific scan pattern. In the specific scan pattern, at least the fovea of the fundus is scanned. As an imaging result of the specific scan pattern, not only axial length information of the subject's eye but also an OCT image (e.g., a tomographic image) at the scanned position may be acquired. The axial length information indicates a distance from a corneal apex to one point on the fundus (preferably, the fovea). Various techniques have been proposed as techniques for acquiring axial length information by OCT. For example, measurement can be performed by appropriately using any of various techniques known as techniques for measuring an ocular axis by an OCT apparatus. For example, axial length information of the subject's eye can be calculated and acquired based on a working distance and depth position information of a retinal surface acquired via the OCT optical system. At this time, a distance between an irradiation position on the cornea and an irradiation position on the retina, which are irradiation positions of the measurement light, can be obtained as the axial length. Note that the working distance is a distance between the apparatus and the subject's eye in a front-rear direction. The working distance may be a fixed value predetermined for each apparatus, or may be a measured value. For example, a measured value may be acquired based on position information of the examination unit in a working-distance direction detected based on an anterior-segment observation image. For a more specific technique, reference is made to, for example, JP 2023-18548 A by the present applicant, which is incorporated herein by reference.

Further, in an examination of the subject's eye by the specific scan pattern, the control unit may restrict a change of an imaging position based on an operation input from an examiner. For example, either one of, or preferably both of, a presentation position of a fixation lamp and a scan position of the measurement light on the fundus is restricted. Accordingly, the axial length information is appropriately acquired.

In addition, in the third embodiment, when analyzing another scan pattern other than the specific scan pattern, for which skipping based on an operation input is not restricted in the combo examination, the analysis processing unit may perform analysis using at least the axial length of the subject's eye acquired based on the specific scan pattern.

In addition, the analysis processing unit may include a first database and a second database. The first database is a first database in which layer thickness information of the fundus regarding a plurality of eyes having a long axial length whose axial length value exceeds a reference is stored. The second database is a database in which thickness information of retinal layers regarding a plurality of subject's eyes having an axial length whose axial length value is within a reference range is stored. In analysis processing for the other scan pattern, the analysis processing unit may refer to at least one of the first database or the second database according to the axial length acquired as an examination result of the subject's eye by the specific scan pattern, and thereby may acquire analysis information regarding layer thickness information of the fundus of the subject's eye. Here, for example, the first database may be a database for long-axial-length eyes, and the second database may be a database for subject's eyes having an axial length different from that of the first database by at least axial length. For example, as the first database and the second database, a long-axial-length database, a normal-eye database, and the like are used. Accordingly, regardless of the axial length of the subject's eye, an analysis result of layer thickness can be appropriately obtained.

It is desirable that the control unit set the execution order of each scan pattern in the combo examination such that the order of the specific scan pattern comes before the other scan patterns. For example, even when analysis by each scan pattern is performed immediately after imaging, since the axial length has been measured in advance, appropriate measurement results and analysis results can be obtained.

### In the Case of OCT+Perimeter

Next, a description is given of a case where the examination unit includes an OCT optical system that acquires OCT data of fundus tissue of a subject's eye, and a visual field examination optical system that performs a visual field examination by irradiating each position on the fundus of the subject's eye with stimulus light.

In this case, the control unit continuously executes, as a combo examination, at least a visual field examination after fundus OCT. The control unit is capable of accepting an operation input for skipping an examination whose turn has arrived during the combo examination. The imaging result of OCT imaging is used to set an initial value of a stimulus threshold in the visual field examination in combo imaging. By reliably using the imaging result of OCT imaging to set the initial value of the stimulus threshold, the examination time and the burden on the subject are significantly reduced.

### In the Case of OCT+Frontal Imaging Device

Also, a description is given of a case where the examination unit includes an OCT optical system that acquires OCT data of fundus tissue of a subject's eye, and a frontal imaging optical system that captures a frontal image of the fundus of the subject's eye.

In this case, the control unit executes, as a combo examination, OCT imaging of the fundus at least successively after capturing a frontal image. The control unit is also capable of accepting an operation input for skipping an examination whose turn has come during the combo examination. The result of capturing the frontal image is used, in OCT imaging, to determine a scan position around a feature portion detected on the frontal image. For example, by analyzing and processing the frontal image, predetermined tissue or a lesion on the fundus may be detected, and a scan position in OCT imaging may be determined with reference to the position of the detected predetermined tissue or lesion. As a technique for detecting the predetermined tissue or lesion, various image-processing methods are known and can be selected as appropriate. By using the result of capturing the frontal image, in OCT imaging, to determine the scan position around the feature portion, OCT data for predetermined tissue or a lesion on the fundus can be reliably captured.

### Embodiment

Next, an ophthalmic imaging apparatus 1, which is an example of the ophthalmic apparatus according to the embodiment, will be described.

The ophthalmic imaging apparatus 1 captures an OCT image of the subject's eye E. Furthermore, as a two-dimensional reflection image of the fundus Er, at least a color fundus image is captured.

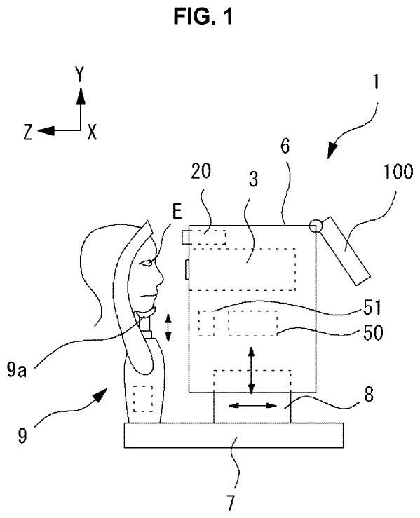

FIG. 1 is an external view of the ophthalmic imaging apparatus 1. In the present example, the ophthalmic imaging apparatus 1 includes an imaging unit 3, a base 7, a drive unit 8, a face support unit 9, a face imaging camera 20, a controller 50, and a touch panel display (hereinafter, referred to as a touch panel) 100. In the following description, for convenience, a unit in which the imaging unit 3 is installed is referred to as an apparatus main body. In the present example, the base 7, the drive unit 8, the face support unit 9, the face imaging camera 20, the controller 50, and the touch panel 100 are parts of the apparatus main body. However, the controller 50 and the touch panel 100 may be units separate from the apparatus main body. The base 7, the drive unit 8, and the face support unit 9 may be omitted as appropriate in the apparatus main body.

In the present example, the drive unit 8 moves the imaging unit 3 on the drive unit 8 in each of the X-, Y-, and Z-directions with respect to the subject's eye E. The drive unit 8 includes actuators for moving the imaging unit 3 in each movable direction, and is driven based on a control signal from the controller 50. The face support unit 9 supports a subject's face. The face support unit 9 is fixed to the base 7. The face support unit 9 includes a chin rest 9a. The chin rest 9a is movable in a vertical direction, thereby adjusting a height of the subject's eye E in accordance with an eye level of the apparatus.

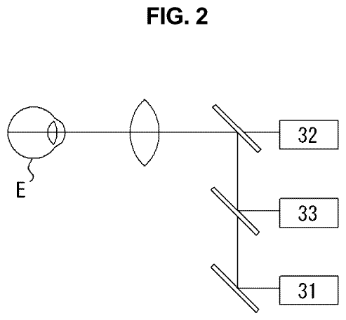

The imaging unit 3 is an imaging unit in the embodiment. The imaging unit 3 mainly includes the optical system illustrated in FIG. 2. As illustrated in FIG. 2, the ophthalmic imaging apparatus 1 includes an OCT optical system 31, a front imaging optical system 32, and an anterior-segment observation optical system 33. In the present embodiment, as illustrated in FIG. 2, the three optical systems share an objective optical system and are coaxialized by a beam splitter/combiner (e.g., a half mirror and a dichroic mirror).

The face imaging camera 20 captures an image of the subject's face. The controller 50 identifies the position of the subject's eye E from the captured face image and drives and controls the drive unit 8, thereby aligning the imaging unit 3 with the identified position of the subject's eye E.

### OCT Optical System

The OCT optical system 31 is used to capture an OCT image of the fundus Er. The OCT image may be a two-dimensional tomographic image or may be a three-dimensional image. In addition, as the OCT image, an intensity OCT image may be acquired, or a motion-contrast image may be acquired.

In the present embodiment, the OCT optical system 31 scans measurement light on tissue of the subject's eye E using a plurality of scan patterns. As an optical scanner for scanning the measurement light, for example, the OCT optical system 31 may include two galvanometer mirrors. Instead of the galvanometer mirrors, for example, MEMS, an acousto-optic element, and the like may also be used.

The OCT optical system 31 includes at least a detector, and the detector detects a spectral interference signal between return light from tissue due to measurement light and reference light. An OCT image is generated by the controller 50 performing predetermined processing on the spectral interference signal.

For further detailed configuration of the OCT optical system 31, refer to, for example, “JP 2011-147609 A filed by the present applicant, which is incorporated herein by reference.

### Front Imaging Optical System

The front imaging optical system 32 acquires, as a captured image, a two-dimensional reflection image of a fundus based on fundus reflected light. The front imaging optical system may be a scanning optical system or a non-scanning optical system. Examples of the scanning optical system include a spot-scan type optical system and a line-scan type optical system. In the spot-scan type optical system, spot-shaped imaging light is scanned two-dimensionally on the fundus. In the line-scan type optical system, line-shaped imaging light is scanned in one direction. Further, an example of the non-scanning optical system includes an optical system of a general fundus camera, etc. For further detailed configuration of the front imaging optical system 32, refer to, for example, “JP 2019-118721 A,” “JP 2019-141737 A,” and “JP 2008-006105 A,” filed by the present applicant, which are incorporated herein by reference.

### Anterior Segment Observation Optical System

The anterior segment observation optical system 33 captures an image of the anterior segment of the subject's eye E and acquires the image as an anterior segment observation image. In the present embodiment, the anterior segment observation optical system 33 illuminates the anterior segment with infrared light as observation light, and a front image of the anterior segment is acquired as the anterior segment observation image based on reflected light. The observation image of the anterior segment is used for alignment and follow-up control (tracking) of the imaging unit 3 with respect to the subject's eye E during fundus imaging.

### Controller

The controller 50 is a processing device (processor) that performs control processing of each unit and arithmetic processing. The controller 50 includes a CPU, a RAM, a ROM, and the like. Also, for convenience, the controller 50 is assumed to perform image processing of various images obtained by the ophthalmic imaging apparatus 1. In other words, the controller 50 also serves as an image processing unit.

The controller 50 is electrically connected to each unit such as the OCT optical system 31, the drive unit 8, the front imaging optical system 32, the anterior segment observation optical system 33, the face imaging camera 20, the storage unit 51, and the touch panel 100.

The storage unit 51 may be a non-transitory storage medium capable of retaining stored contents even when supply of power is interrupted. For example, the storage unit 51 stores various control programs, fixed data, and the like. Also, for example, captured images captured by the ophthalmic imaging apparatus 1 are stored in the storage unit 51. The captured images may be transferred to an external storage device (for example, a storage device connected to the controller 50 via LAN and WAN).

Also, the storage unit 51 stores an analysis program, a normal-eye database, and a long-axial-length normal-eye database. The analysis program is a program for performing layer thickness analysis on OCT data of a fundus. In the layer thickness analysis, either the normal-eye database or the long-axial-length normal-eye database is used, and information indicating a comparison result between measurement values (actual measured values) based on the OCT data and normal-eye data is output as an analysis result. Here, the normal-eye database stores information representing a distribution of layer thicknesses in a plurality of normal eyes having an axial length equal to or less than a threshold. The long-axial-length normal-eye database stores information representing a distribution of layer thicknesses in a plurality of normal eyes having an axial length exceeding the threshold. In the analysis processing, the normal-eye database and the long-axial-length normal-eye database are selectively used depending on axial length data of a subject's eye. At this time, as a value of the axial length data of the subject's eye, a measured value measured by the apparatus may be acquired.

### Touch Panel Display

The touch panel 100 serves both as a monitor and as an operation input unit in the ophthalmic imaging apparatus 1. Various images are displayed on the touch panel 100. In addition, a graphical user interface (GUI) is displayed to accept an examiner's operations on UI elements (widgets). Various touch operations are input via the touch panel 100.

### Detailed Screen Configuration

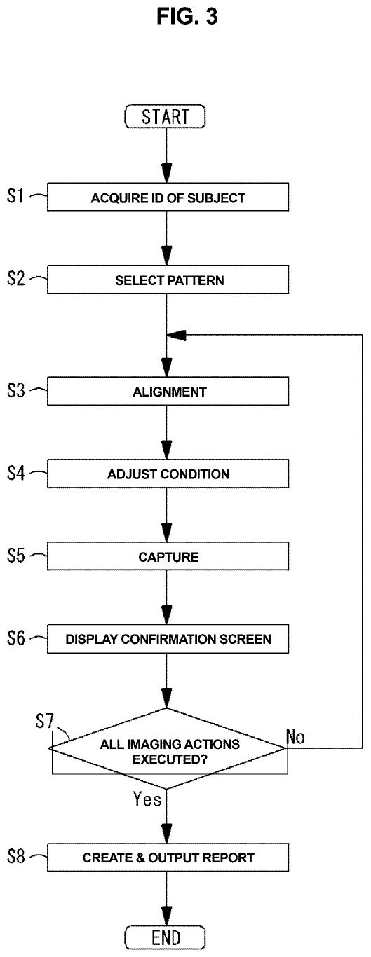

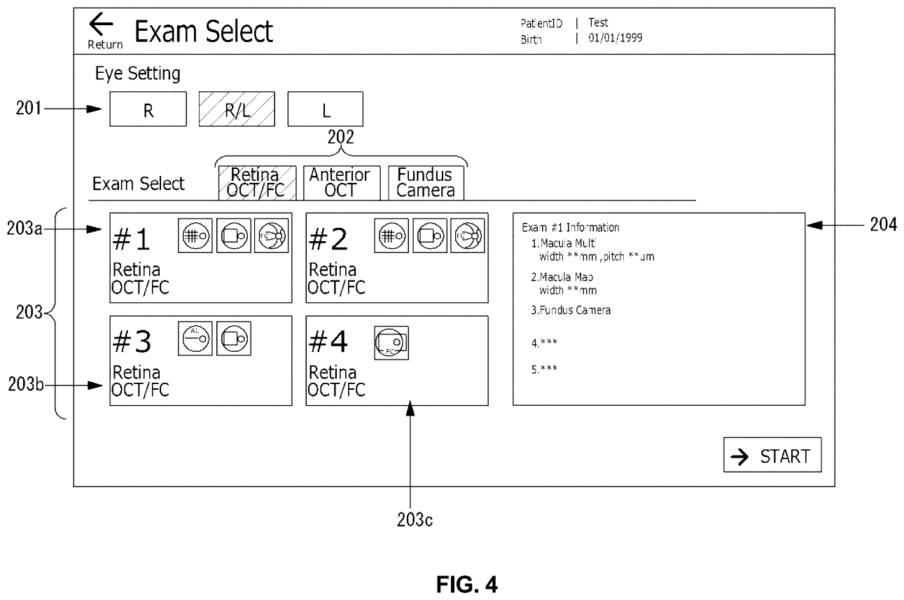

A graphical user interface (GUI) used for imaging in the OCT apparatus 1 will be described along the flow of operation of the ophthalmic imaging apparatus 1 in the present embodiment. FIG. 3 illustrates a flowchart representing the flow of operation. In addition, screens displayed on the touch panel 100 at each timing in the operation are shown in FIGS. 4 to 22.

First, new registration or retrieval of subject identification information (ID, name, age, etc.) is performed (S1). For example, the identification information is input by reading a barcode in which the subject identification information is described with a barcode reader connected to the apparatus, or by directly inputting the information via a keyboard. When imaging a subject whose identification information has already been registered, the identification information may be retrieved by displaying a subject list and selecting any corresponding one from the list.

### Examination Pattern Selection

Next, the controller 50 accepts a selection operation for an examination pattern (S2). In this case, an examination selection screen shown in FIGS. 4 to 6 is displayed. Based on operation inputs via the examination selection screen, imaging details including an imaging method are determined.

As illustrated in FIGS. 4 to 6, the examination selection screen displays, as UI elements, an eye selection button 201, a mode switching tab 202, an examination selection button 203, and a text box 204.

The eye selection button 201 is a UI element for selecting, as an eye to be imaged, one of a right eye, a left eye, and both eyes. In the example of FIGS. 4 to 6, three buttons corresponding to the right eye, the left eye, and both eyes, respectively, are provided. When the right eye or the left eye is selected as the eye to be imaged, the examined eye E on the selected side is imaged using a separately selected combo pattern (examination pattern). When both eyes are selected as the eye to be imaged, after one eye is imaged using a separately selected combo pattern, the opposite eye is imaged using a similar combo pattern.

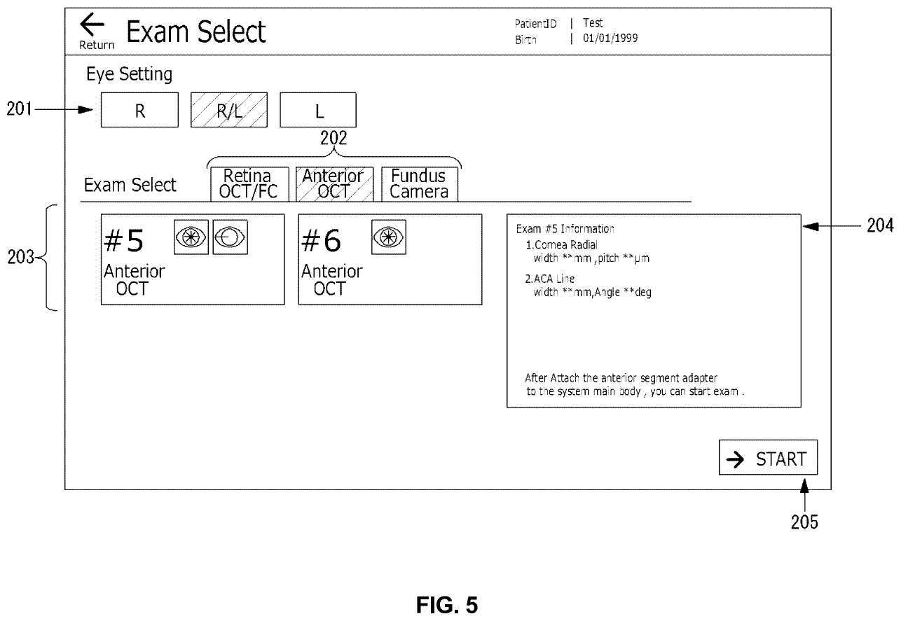

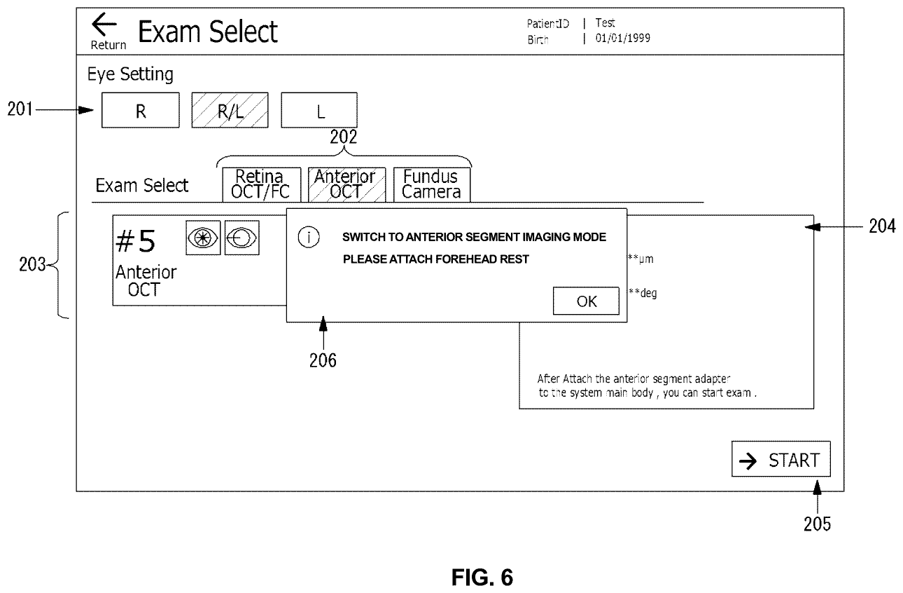

The mode switching tab 202 is operated to switch an imaging mode. In the present embodiment, one of three imaging modes is selected: an OCT/FC mode (see FIG. 4), an anterior-segment OCT mode (see FIGS. 5 and 6), and an FC mode (not illustrated). The OCT/FC mode is selected when an OCT image of a fundus is to be captured. In the OCT/FC mode, capturing a front image of the fundus (any of a color fundus image, an infrared image, and an autofluorescence image) can also be performed in combination with capturing an OCT image. The anterior-segment OCT mode is selected when an OCT image of an anterior segment is to be captured. The FC mode is selected when a fundus front image is to be captured without combining OCT imaging.

The examination selection button 203 is a UI element for selecting a combo pattern. Each combo pattern includes one or more imaging operations. A combo pattern including a plurality of imaging operations is provided to successively perform the plurality of imaging operations on one examined eye E. For example, as an example of a combo pattern including a plurality of imaging operations, a pattern combining a plurality of scan patterns is possible. Each scan pattern specifies a position on tissue at which an OCT image is acquired and a number of OCT images to be acquired. Examples of the types of scan patterns include a line scan, a cross scan, a multi-scan, a radial scan, and a raster scan (also referred to as a map scan). Further, for each type, a plurality of scan patterns having mutually different positions on tissue and scan lengths may be provided. Also, for example, as an example of a combo pattern including a plurality of imaging operations, a pattern combining a plurality of imaging operations having different image types is possible. For example, the pattern may be a pattern in which an OCT image and a fundus front image are captured successively, or may be a pattern in which an intensity OCT image and a motion contrast image are captured successively.

In the present embodiment, for convenience of explanation, when imaging is performed with a line pattern with the macula as an imaging position, measurement of axial length data is performed together with the imaging. However, the scan pattern in imaging performed together with measurement of axial length data may be any scan pattern other than the line pattern. For example, the frequency of use of a layer thickness map and a chart generated by analyzing and processing three-dimensional OCT data using a normal-eye database and a long-axial-length normal-eye database is high. Therefore, it is useful if axial length data is measured together with a map scan (raster scan). However, in the case of a map scan, since scan lines passing through the macula constitute only a part, for example, each B-scan acquired by the map scan and an anterior-segment observation image at the same timing as each B-scan may be acquired in advance, and axial length data may be acquired based on B-scan OCT data in which the macula is detected and an anterior-segment observation image acquired at the same timing.

In the present embodiment, for each screen, a maximum of four buttons are arranged as examination selection buttons 203. One combo pattern is associated with each button. Although details will be described later, the correspondence relationship between the buttons and the combo patterns can be set and changed in advance via a setting screen described later.

As illustrated in FIGS. 4 to 6, each button is provided with identification information corresponding to each imaging operation included in the combo pattern. In the present embodiment, pictorial symbols are used as the identification information. In the case of a combo pattern including a plurality of imaging operations, a plurality of pictorial symbols corresponding to the respective imaging operations are provided.

For example, in FIG. 4, the button 203a indicated by #1 is provided with pictorial symbols respectively corresponding to “macular multi” and “macular map,” which are scan patterns in OCT imaging, and is further provided with a pictorial symbol corresponding to imaging of a fundus frontal image. The button 203b indicated by #3 is provided with pictorial symbols respectively corresponding to “macular line” and “macular map,” which are scan patterns in OCT imaging. The button 203c indicated by #4 is provided with a pictorial symbol corresponding to a continuous pattern. The continuous pattern is a scan pattern in which a wide-area map (retinal map) including the macula and the optic disc and a fundus frontal image are captured in a series.

For example, as illustrated in FIG. 4, the pictorial symbols related to fundus OCT may indicate a pattern shape (scan locus) in each scan pattern. The examiner can easily understand, via pictorial symbols indicating the pattern shapes of the respective scan patterns, the combination of scan patterns included in the combo pattern. The pictorial symbol corresponding to imaging of a fundus frontal image may schematically represent the fundus frontal image. Thus, in the present embodiment, by arranging one or more pictorial symbols corresponding to the respective imaging operations on each button, the content of the combo pattern corresponding to each button is indicated.

When any of the examination selection buttons 203 is selected, details of the combo pattern corresponding to the selected button are displayed in the text box 204 as text information. That is, imaging conditions (scan conditions) for each imaging operation included in the combo pattern are presented for each imaging operation.

By operating the start button 205 while any of the examination selection buttons 203 is selected, an examination pattern is determined, and the screen transitions from the examination selection screen to the next screen (in this embodiment, an alignment screen).

In this embodiment, as illustrated in FIGS. 4 to 6, when the imaging mode is switched based on operation of the mode switching tab 202, the examination selection buttons 203 are also changed in conjunction with the imaging mode. For example, as illustrated in FIG. 5, in an anterior-segment OCT mode, as the examination selection buttons 203, buttons on which graphical symbols indicating scan patterns in anterior-segment OCT are arranged are displayed.

In this embodiment, imaging of anterior-segment OCT is enabled by attaching an anterior-segment adapter (not shown) to an examination window of the imaging unit 3. For details of the anterior-segment adapter, refer to, for example, “JP 2011-147609 A” filed by the present applicant, which is incorporated herein by reference.

Therefore, when a button corresponding to a combo pattern including a scan pattern of anterior-segment OCT is selected, a message indicating that attachment of an anterior-segment adapter is required is displayed in the text box 204. In this case, for example, the controller 50 may, in parallel with displaying the examination selection screen, detect attachment and detachment of the anterior-segment adapter. When the anterior-segment adapter is not attached, the controller 50 may disable an operation on the start button 205 even if the examination selection button 203 currently selected corresponds to a combo pattern including a scan pattern of anterior-segment OCT. The message indicating that attachment of the anterior-segment adapter is required may be output when an operation on the start button 205 is input and attachment of the anterior-segment adapter is not detected. In the present embodiment, when capturing anterior-segment OCT, a forehead rest for working-distance adjustment is attached to the face support unit 9 together with the anterior-segment adapter. Triggered by detection of attachment of the anterior-segment adapter, as illustrated in FIG. 6, a message prompting attachment of the forehead rest may further be displayed by a pop-up window 206. This can suitably suppress forgetting to attach the forehead rest when capturing anterior-segment OCT.

### Alignment

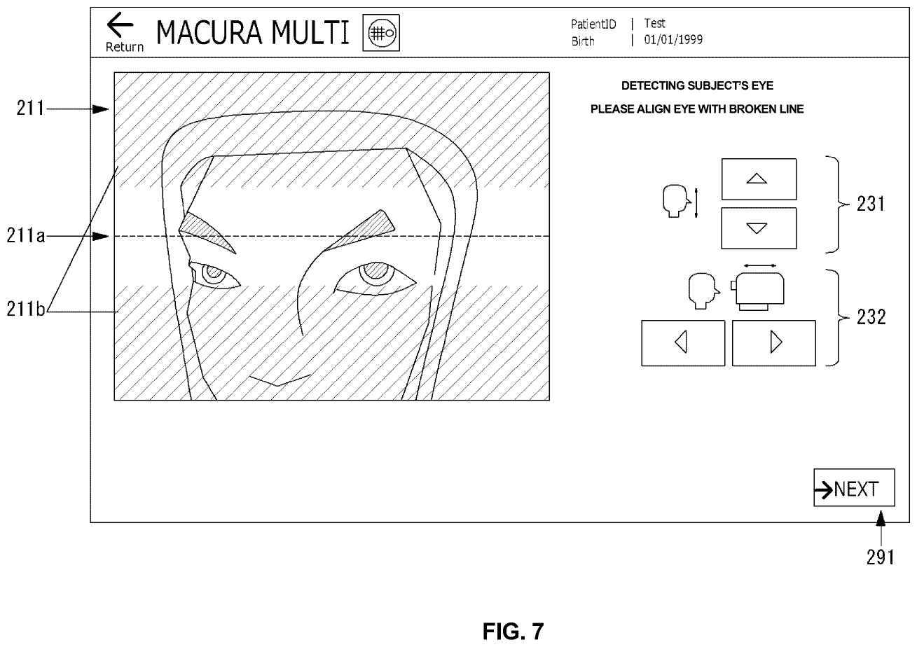

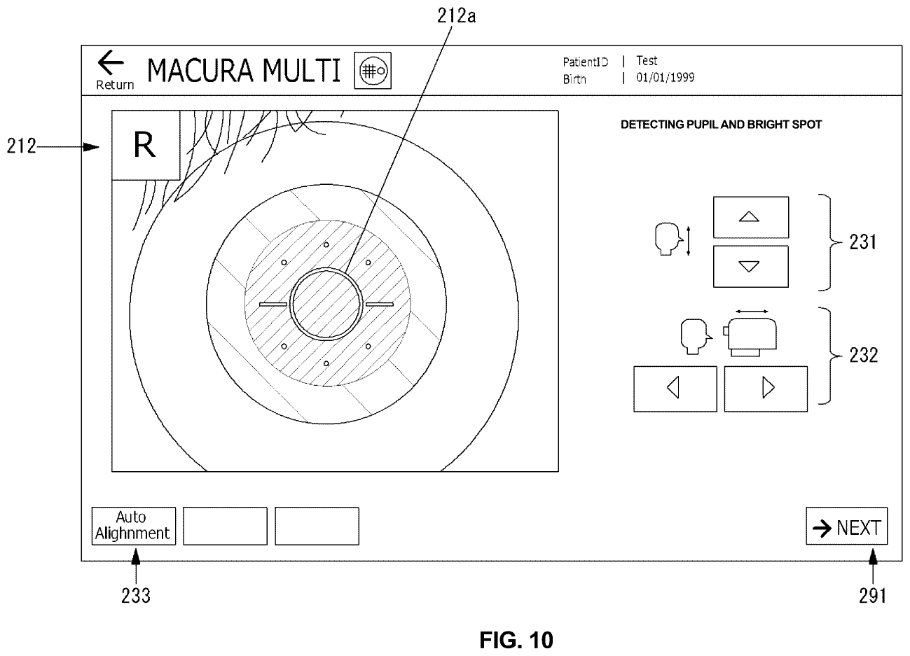

On the examination selection screen, when the examination selection button 203 is selected and the start button 205 is further operated, the display transitions to an alignment screen. The alignment screen is illustrated in FIGS. 7 and 10. In this state, alignment adjustment is executed (S3). The controller 50 sequentially acquires a face image and an anterior-segment observation image via the face imaging camera 20 and the anterior-segment observation optical system 33, respectively.

On the alignment screen shown in FIGS. 7 and 10, at least an observation image for alignment is displayed. The observation image may be either a face image 211 (see FIG. 7) acquired via the face imaging camera 20 or an anterior-segment observation image 212 (see FIG. 10) acquired via the anterior-segment observation optical system 33. In parallel with the display of the alignment screen, the controller 50 adjusts the position of the imaging unit 3 relative to the subject's eye E, to a position at which the subject's eye E can be imaged, based on the observation image. In the present embodiment, the position of the imaging unit 3 and the position of the chin rest 9a in the face support unit 9 are adjusted from respective predetermined initial positions to a state in which the optical axis of the imaging unit 3 coincides with the subject's eye E and further becomes an appropriate working distance.

On the alignment screen shown in FIGS. 7 and 10, a chin-rest height adjustment button 231 and a forward/backward adjustment button 232 are further displayed. The chin-rest height adjustment button 231 includes a pair of buttons corresponding to upward and downward directions. By operation of the chin-rest height adjustment button 231, the controller 50 moves the chin rest 9a of the face support unit 9 in an up-down direction. The forward/backward adjustment button 232 is operated to change the position of the imaging unit 3 in a forward/backward direction. The forward/backward adjustment button 232 includes a pair of buttons corresponding to forward and backward directions. By operation of the forward/backward adjustment button 232, the controller 50 moves the imaging unit 3 in the forward/backward direction.

In the present embodiment, first, as shown in FIG. 7, a face image 211 obtained via the face imaging camera 20 is displayed as an observation image.

The controller 50 analyzes the face image 211 to detect the position of the subject's eye E. At this time, in the observation image display area, a line 211a indicating a reference height of eye level is displayed at a fixed position. The line 211a is indicated by a broken line in FIG. 7. When the position of the subject's eye E is separated from the line 211a, the examiner operates the chin-rest height adjustment button 231 to bring the subject's eye E closer to the line 211a, whereby the subject's eye E (and the position of the subject's eye E) becomes detectable from the face image 211. The position of the subject's eye E may be acquired as two-dimensional coordinates on the face image 211.

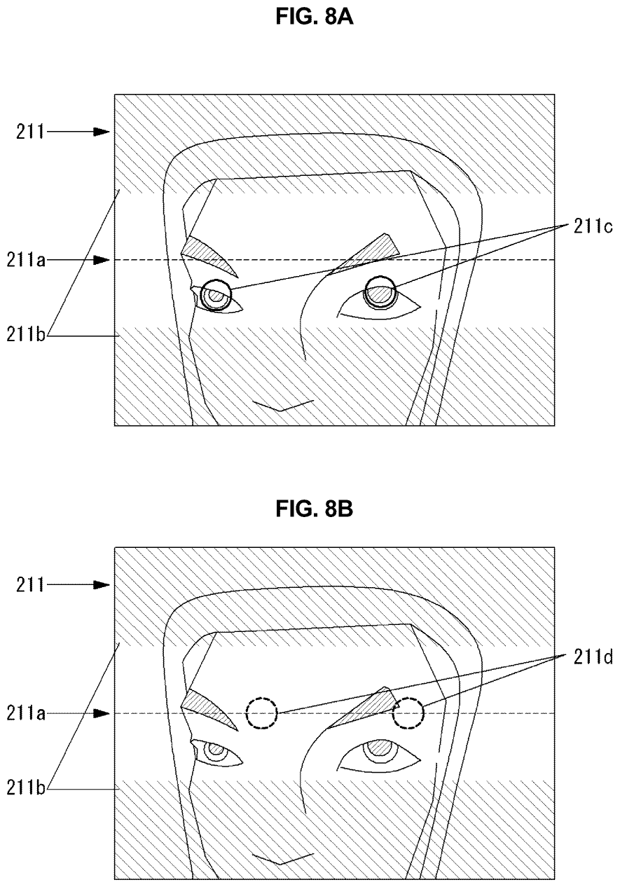

When detection of the subject's eye E is performed on the face image 211, the detection result is displayed. In the present embodiment, FIG. 8A illustrates, as an example, the face image 211 when the detection is successful, and a mark 211c is superimposed on the position of the subject's eye E detected in the face image 211. The mark 211c upon successful detection is, as an example, a green circle formed by a solid line. However, due to erroneous detection, the mark 211c may be superimposed on another position such as an eyebrow. In this case, by touching the correct position of the subject's eye E, the detected position is corrected to the touched position, or to the position of the subject's eye E detected in a peripheral region around the touched position. In this case, a correction mark (not shown) is superimposed on the corrected detection position. The correction mark is displayed in a mode different from that of the mark 211c upon successful detection. As an example, the correction mark is a light-blue circle formed by a solid line. FIG. 8B illustrates, as an example, the face image 211 when the detection is unsuccessful. If the detection is unsuccessful, a mark 211d in a mode different from that upon successful detection is superimposed at a predetermined position. In FIG. 8B, as an example, the mark 211d is superimposed on the line 211a. The mark 211d upon unsuccessful detection is, as an example, a red circle formed by a broken line. Also in this case, similarly to the above, the detected position can be corrected by touching the correct position of the subject's eye E. Further, before detection of the subject's eye E is completed (i.e., during execution of the detection processing), if the position of the subject's eye E on the face image 211 is touched, the detection position of the subject's eye E is set to the touched position, or to the position of the subject's eye E detected in a peripheral region around the touched position. In this case, a correction mark is superimposed on the face image 211 at the set detection position.

Once the position of the subject's eye E becomes detectable based on the face image 211, the controller 50 moves the imaging unit 3 from the initial position to perform automatic alignment. In the present embodiment, when the examiner confirms the detection result of the subject's eye and operates the transition button 291, movement of the imaging unit 3 is started. If detection of the subject's eye has failed, operation on the transition button 291 is invalid unless the detected position is corrected, and movement of the imaging unit 3 is not started. This improves the reliability of automatic alignment.

When movement of the imaging unit 3 is started, the position of the imaging unit 3 is changed in a direction in which an optical axis of the imaging unit 3 (hereinafter referred to as an imaging optical axis) approaches the detected subject's eye E. That is, the position of the imaging unit 3 is adjusted based on the position of the subject's eye E detected based on the face image 211. In the present embodiment, upon completion of the movement, precise alignment of the imaging unit 3 can be started based on an anterior ocular segment observation image acquired via the anterior ocular segment observation optical system 33. Thus, alignment of the imaging unit 3 based on the face image 211 ends.

In the present embodiment, the movable range of the imaging unit 3 in the vertical direction is narrower than the angle of view of the face image 211. Even if the subject's eye E is detected outside the movable range, appropriate alignment cannot be performed only by movement of the imaging unit 3.

In contrast, in the present embodiment, when the face image 211 is displayed as an observation image, a mask 211b is superimposed on the display area of the observation image. As an example, the mask 211b is a uniform semi-transparent image. In the present embodiment, the mask 211b is superimposed on each of upper and lower portions of the display area. The mask 211b indicates that the portion is outside the movable range of the imaging unit 3. More specifically, in the present embodiment, a region of the face image 211 on which the mask 211b is superimposed is outside the movable range (a second range of the embodiment) of the imaging unit 3 when the imaging unit 3 is at a predetermined position (an initial position in the present embodiment). For example, the range over which the mask 211b is to be superimposed can be specified by examining, on the face image 211 acquired when the imaging unit 3 is at the predetermined position, a range that the imaging optical axis cannot reach when the imaging unit 3 is moved. A central portion 211a of the face image 211 that is not overlapped with the mask 211b (a first range of the embodiment) is the movable range of the imaging unit 3. That is, it is a range that the imaging optical axis can reach when the imaging unit 3 is moved. Therefore, in the face image 211, if the mask 211b does not overlap the subject's eye E, appropriate alignment can be performed by moving the imaging unit 3.

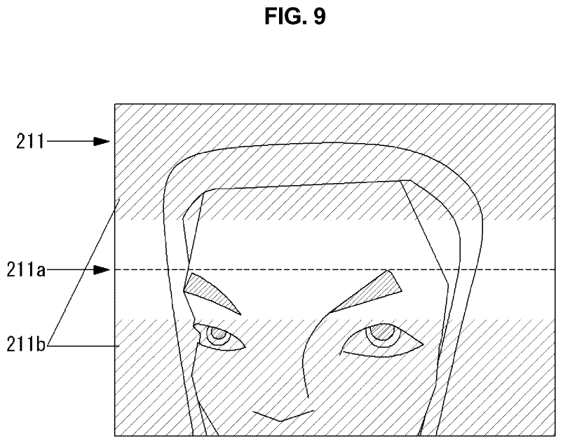

On the other hand, as illustrated in FIG. 9, in the face image 211, when the mask 211b overlaps the subject's eye E, the subject's eye E is located outside the movable range of the imaging unit 3. In the case of FIG. 9, since the position of the subject's eye E is lower than the movable range of the imaging unit 3, the subject's eye E can be positioned within the movable range of the imaging unit 3 by operating the chin-rest height adjustment button 231 in a direction to raise the chin rest 9a. As a result, by moving the imaging unit 3, a positional relationship between the imaging unit 3 and the subject's eye E can be appropriately adjusted. In this manner, since the mask 211b is superimposed on a region in the face image 211 that is outside the movable range of the imaging unit 3, the examiner can easily determine whether height adjustment of the chin rest 9a is necessary. In addition, when the position of the subject's eye E on the face image 211 is outside the movable range of the imaging unit 3, operation of the chin-rest height adjustment button 231 can be appropriately prompted to the examiner.

In the present embodiment, the controller 50 stops movement of the imaging unit 3 when the position of the subject's eye E on the face image 211 is outside the movable range of the imaging unit 3, and moves the imaging unit 3 when the position of the subject's eye E on the face image 211 is within the movable range of the imaging unit 3.

Here, in the present embodiment, the controller 50 controls setting of the position of the subject's eye E on the face image 211 such that the position of the subject's eye E on the face image 211 can be set (detected and corrected in the present embodiment) within the movable range of the imaging unit 3, and cannot be set outside the movable range (i.e., at a position overlapping the mask 211b). In the face image 211, when the mask 211b overlaps the subject's eye E, the examiner can first be prompted to adjust the position of the subject's face by height adjustment of the chin rest 9a, or the like. As a result, the examiner is likely to be able to smoothly proceed with the examination in an appropriate procedure. In particular, in the present embodiment, unless detection of the subject's eye in the central portion 211a that is not overlapped with the mask 211b is successful or a correction position is set, an operation on the transition button 291 is invalid and movement of the imaging unit 3 is not started, and thus, when the position of the subject's eye E on the face image 211 is outside the movable range of the imaging unit 3, alignment adjustment is likely to be suppressed from being performed in an inefficient procedure.

As described above, in the present embodiment, when the imaging unit 3 is at a predetermined position (e.g., an initial position), the mask 211b is superimposed outside the movable range of the imaging unit 3; however, the present disclosure is not necessarily limited thereto, and when the face image 211 is displayed during movement of the imaging unit 3, the position of the mask 211b on the face image may be changed in accordance with the position of the imaging unit 3.

In the present embodiment, a case has been described in which the examiner himself/herself determines whether or not the position is outside the movable range of the imaging unit 3 by checking the observation image and adjusts the height of the chin rest based on the examiner's operation; however, the present disclosure is not necessarily limited thereto. For example, the controller 50 may determine whether or not the position of the subject's eye E detected from the face image 211 is outside the movable range of the imaging unit 3, and furthermore, when the position of the subject's eye E is outside the movable range of the imaging unit 3, may automatically adjust the height of the chin rest, or may output, via the user interface, guide information prompting a height adjustment operation of the chin rest 9a. However, when detecting the subject's eye E from the face image 211, erroneous detection may occur, or detection may fail. In contrast, as in the present embodiment, indicating, on the observation image in which the face image is displayed, an area outside the movable range of the imaging unit 3 and prompting the examiner to intervene may, in some cases, facilitate smooth progress of the examination.

After the transition button 291 is operated and alignment of the imaging unit 3 based on the face image 211 is completed, an anterior-segment observation image 212 is displayed as an observation image (see FIG. 10).

In the present embodiment, when the auto-alignment button 233 is selected, the position of the imaging unit 3 is automatically moved with respect to the subject's eye E based on the anterior-segment observation image 212. In the present embodiment, after projecting an alignment index and adjusting the positional relationship in each of the X-, Y-, and Z-directions, furthermore, with respect to the X-and Y-directions, adjustment is made such that the imaging optical axis coincides with the pupil center. In the present embodiment, an indicator 212a is superimposed and displayed on the anterior-segment observation image 212. The mode of the indicator 212a changes in accordance with an alignment state. Thereby, the alignment state is indicated. In the present embodiment, based on a touch operation on the anterior-segment observation image 212, it is possible to adjust a position, in the X-and Y-directions, of the imaging optical axis with respect to the subject's eye E. For example, when an area on the anterior-segment observation image 212 in a direction in which the indicator 212a is desired to be moved is touched, the imaging unit 3 is moved in accordance with the direction. Further, the present disclosure is not necessarily limited thereto; the imaging unit 3 may be moved such that the center (imaging optical axis) of the indicator 212a is moved to the touched position, or pupil detection may be performed with the touched position as a center and the imaging unit 3 may be moved such that the center (imaging optical axis) of the indicator 212a is moved to the center of a detected pupil region.

### Adjustment of Imaging Conditions

After completion of alignment, imaging conditions are adjusted (S4). In the present embodiment, after completion of alignment, the display transitions from an alignment screen to an observation screen.

### Combo Pattern #1

First, a case will be described where, on the examination selection screen shown in FIG. 4, the button 203a is selected in the OCT/FC mode. In this case, the operation is such that an OCT image is captured by macular multi and macular map, and further, a fundus front image is captured.

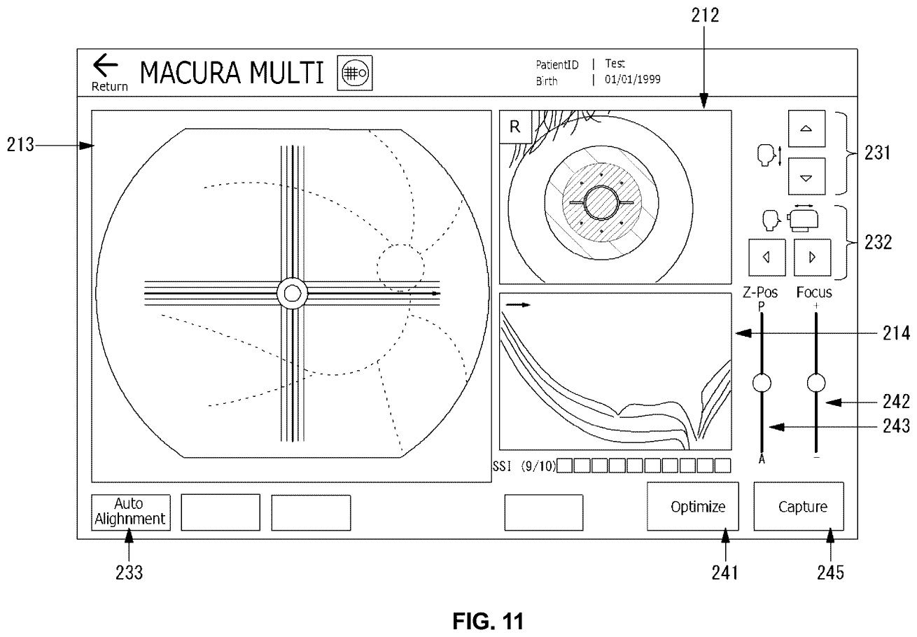

In this case, capturing an OCT image using the scan pattern of macular multi is performed first. FIG. 11 shows an observation screen displayed in this case. In the present embodiment, a configuration of the observation screen used when capturing an OCT image will be described with reference to FIG. 11. As shown in FIG. 11, in the present embodiment, the observation screen used when capturing an OCT image displays, on the screen, a fundus front image 213, a tomographic image 214, a detection condition controller, and a Capture button 245. Before execution of capture, the fundus front image 213 and the tomographic image 214 displayed on the observation screen may be observation images acquired in real time. In addition, on the fundus front image 213, a graphic indicating the scan pattern is displayed. With the graphic, the examiner can grasp an imaging position of the OCT image.

The imaging-condition controller is operated to adjust detection conditions of the OCT optical system 31 and imaging conditions of the front-imaging optical system 32. The detection conditions are any of various conditions related to a detection capability for eye tissue, and specific examples include an optical path length difference (hereinafter, OPL), focus, detection sensitivity, a polarization state, and the like. Examples of the imaging conditions include, for example, focus, brightness (any of light amount, gain, and exposure time), contrast, and the like.

In the present embodiment, an Optimize button 241, a focus controller 242, and an OPL controller 243 are arranged as the imaging-condition controller.

When the Optimize button 241 is operated, the controller 50 executes adjustment processing of the OCT optical system 31 and the front imaging optical system 32. In the present embodiment, as an example, at least an OPL, focus, and polarization in the OCT optical system 31 and a focus of the front imaging optical system 32 are adjusted to predetermined states. The focus of the OCT optical system 31 and the front imaging optical system 32 may be adjusted based on a fundus observation image (fundus front image 213). In this case, a focus index may be projected onto the fundus, and the focus may be adjusted based on the focus index reflected in the fundus observation image. The result of the adjustment processing is reflected, for example, in image quality, image position, and the like of the fundus front image 213 and the tomographic image 214. When the user checks the fundus front image 213 and the tomographic image 214 and determines that the adjustment has failed, the user can further operate the focus controller 242 and the OPL controller 243 to attempt manual adjustment.

The focus controller 242 and the OPL controller 243 are operated to manually adjust detection conditions and imaging conditions, respectively. In the present embodiment, the focus controller 242 and the OPL controller 243 are sliders, and the position of a “knob” is moved based on a drag operation or the like. The respective imaging conditions and detection conditions are set according to the position of the knob. For example, the slider of the focus controller 242 and/or the OPL controller 243 is operated according to a depth position of a layer region to be imaged with higher sensitivity.

Additionally, in the present embodiment, an anterior-segment observation image 212, a chin-rest height adjustment button 231, a longitudinal adjustment button 232, and an auto-alignment button 233 are also displayed on the observation screen. Thus, even after transition to the observation screen, the alignment state can be checked. When the position of the subject's eye E has moved from a proper alignment position, alignment adjustment can be performed automatically or manually.

### Capture

The Capture button 245 accepts an operation serving as a trigger to start imaging (capture). When the Capture button 245 is operated, an imaging scan is performed in a predetermined scan pattern (in FIG. 11, a macular multi scan pattern) (S5).

### Display of Confirmation Screen

When imaging is completed, the process transitions to a confirmation screen. The confirmation screen is a screen for confirming imaging results. In the present embodiment, the output contents of a report can also be selected via the confirmation screen.

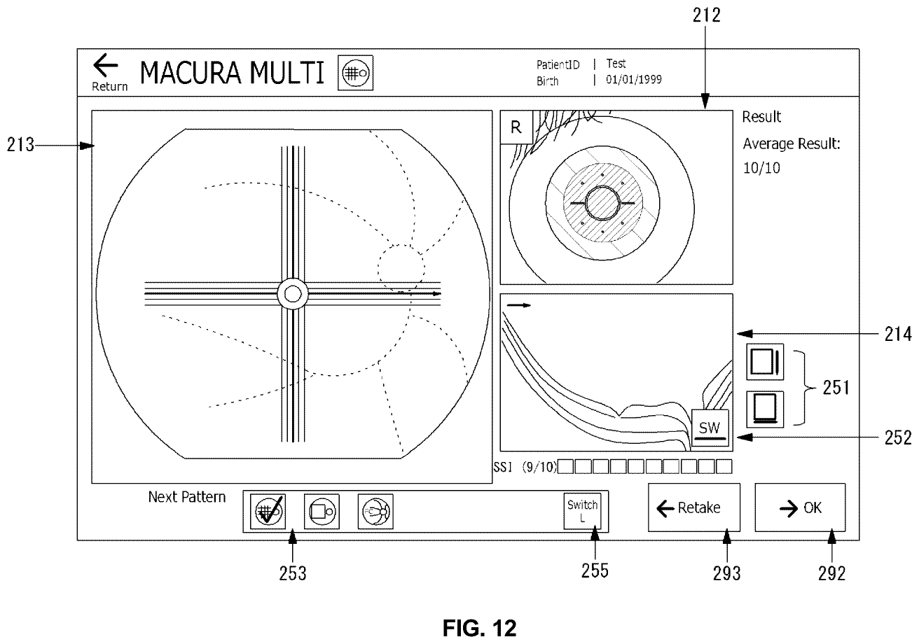

For example, when imaging using a macular multi scan pattern is completed, a screen shown in FIG. 12 is displayed as the confirmation screen. In the present embodiment, on the confirmation screen, in addition to a tomographic image 214 obtained by an imaging scan, an anterior-segment observation image 212 and a fundus frontal image 213 are displayed. The anterior-segment observation image 212 and the fundus frontal image 213 displayed on the confirmation screen may be still images acquired simultaneously with the imaging scan. Through the anterior-segment observation image 212 and the fundus frontal image 213, it is possible to confirm an alignment state during imaging and whether imaging conditions are appropriate.

When tomographic images 214 are captured for a plurality of scan lines, the tomographic image 214 displayed on the confirmation screen can be switched to an image captured for another scan line. In the present embodiment, a scan line change button 251 is arranged on the confirmation screen. Each time the scan line change button 251 is operated, the tomographic image 214 displayed on the confirmation screen is switched to an image on another scan line in accordance with a predetermined order. The tomographic image 214 displayed on the confirmation screen is used as a representative image among the plurality of scan lines. The representative image is an image to be output in the report among the tomographic images captured for the plurality of scan lines. In the case of a macular multi scan pattern, one representative image is set for each scan line in each of the X and Y directions. However, the number of representative images and the direction of scan lines can be appropriately determined for each scan pattern.

In addition, by operating the switch button 252, the tomographic image displayed on the screen is switched between the scan line in the X direction and the scan line in the Y direction. In addition, by operating a button (not shown), the tomographic image 214 is displayed in an enlarged manner.

An OK button 292 and a Retake button 293 are further arranged on the confirmation screen. The Retake button 293 is operated to perform re-imaging. In the present embodiment, when the Retake button 293 is operated, the most recent imaging result is discarded and a transition is made to the observation screen. In this case, imaging is performed with the same scan pattern as that of the most recent imaging.

When the OK button 292 is operated, the imaging result is stored. Further, when the selected combo pattern includes an imaging operation that has not been performed, a transition is made to the observation screen. In this case, the scan pattern or the type of image to be captured is changed to that of the next order.

The progress status (completion status) of the imaging operations included in the selected combo pattern is shown in the progress display box 253 (progress display region in the embodiment). Specifically, in the progress display box 253, one or more pictograms corresponding respectively to the imaging operations included in the preselected combo pattern are arranged side by side. The pictograms shown in the progress display box 253 are the same as the pictograms displayed on the button selected by the examiner among the examination selection buttons 203. As illustrated in FIG. 12, a check mark is superimposed on the pictogram corresponding to a completed imaging operation. Thus, completed imaging operations and incomplete imaging operations are distinguished from each other. Accordingly, the examiner can readily grasp the progress status of the imaging operations included in the combo pattern via the pictograms.

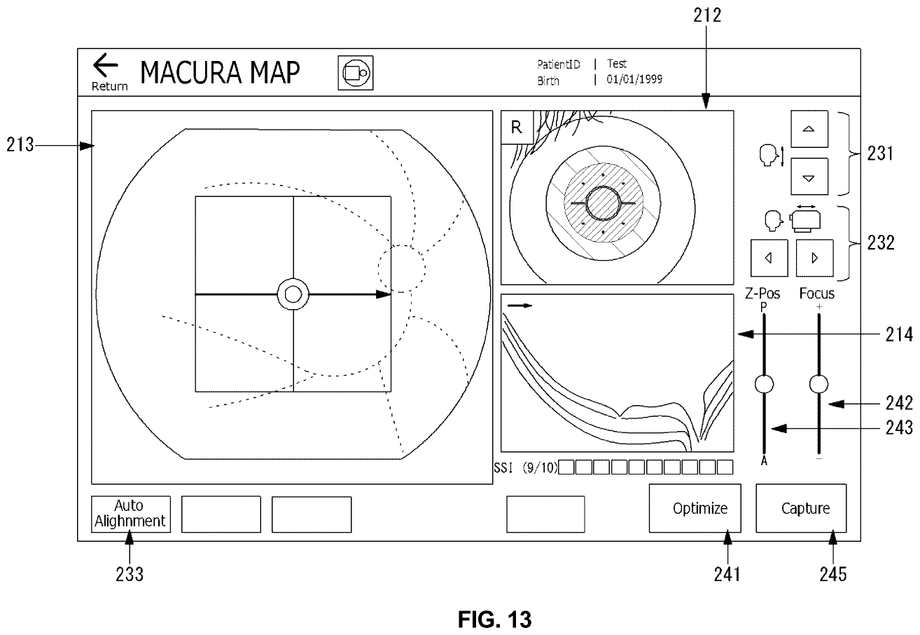

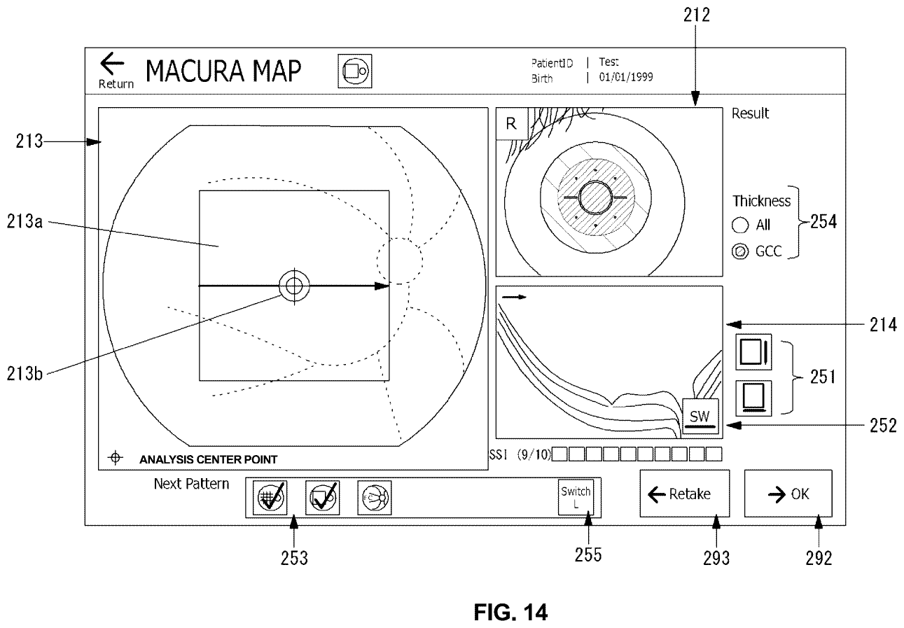

When the OK button 292 is operated on the confirmation screen, if there exists an imaging operation that has not yet been executed (S7: No), the process proceeds to the next imaging operation. In the confirmation screen of FIG. 12, when the OK button 292 is operated, the process proceeds to imaging using a macular map, which is the next imaging operation. At that time, the controller 50 causes the screen to transition from the confirmation screen illustrated in FIG. 12 to the observation screen illustrated in FIG. 13. In the observation screen for capturing a macular map, the graphic superimposed on the fundus front image 213 represents the scan pattern of the macular map. The other points are the same as in FIG. 11. By operating the Capture button 245, imaging using the macular map is executed. As a result, a three-dimensional tomographic image centered on the macula is captured. When imaging is completed, a transition is made to the confirmation screen illustrated in FIG. 14.

In the present embodiment, via the confirmation screen illustrated in FIG. 14, an operation input for changing and setting an analysis region 213a in a three-dimensional tomographic image can be accepted. Specifically, the position of the analysis region 213a can be changed in the X-Y directions via the front image 213. For example, a mark 213b indicating the center position of the analysis region 213a can be moved in accordance with an operation on the touch panel 100. In the present embodiment, the reference position of analysis processing using a normal-eye database is displaced in accordance with the center position. However, the present disclosure is not necessarily limited thereto, and a reference position in the analysis processing may be changeable. Further, when changing the analysis region 213a in a depth direction, an analysis-region change button 254 is operated. As one example, in FIG. 14, as the analysis-region change button 254, a button corresponding to the entire retinal layer(s) and a button corresponding to a GCC (Ganglion Cell Complex) are arranged. By selecting any one of the buttons, a region corresponding to the selected button is set as the analysis region 213a in the depth direction. Note that the GCC is a composite layer region including a retinal nerve fiber layer (RNFL), a ganglion cell layer (GCL), and an inner plexiform layer (IPL).

The analysis region 213a changed or set via the confirmation screen is reflected in analysis processing on an imaging result displayed on the confirmation screen. In the present embodiment, as one example, analysis processing relating to a tissue thickness is performed. A two-dimensional distribution of measured values of tissue thickness and/or a two-dimensional distribution of comparison results between a measured value and a tissue thickness of a normal eye is output as a result of the analysis processing. The analysis processing may be executed, for example, at a timing at which a report on the imaging result is generated, or may be executed at another timing. Further, the content of the analysis processing is not limited to analysis processing relating to layer thickness (details will be described later).

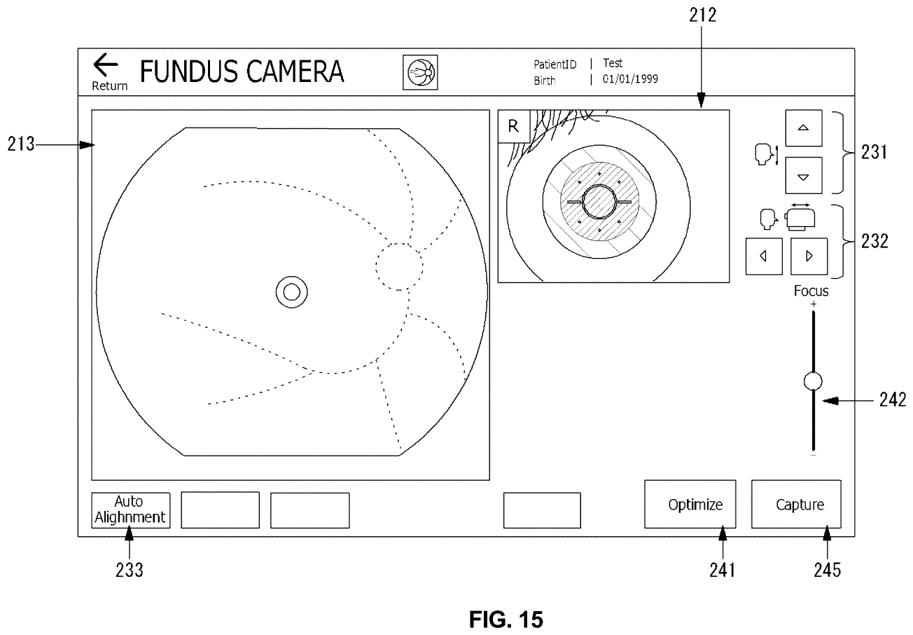

In the confirmation screen of FIG. 14, when the OK button 292 is operated, the process proceeds to capturing a fundus front image, which is the next imaging operation. At that time, the controller 50 transitions the screen from the confirmation screen shown in FIG. 14 to the observation screen shown in FIG. 15. In the following description, as an example, a color image is assumed to be captured.

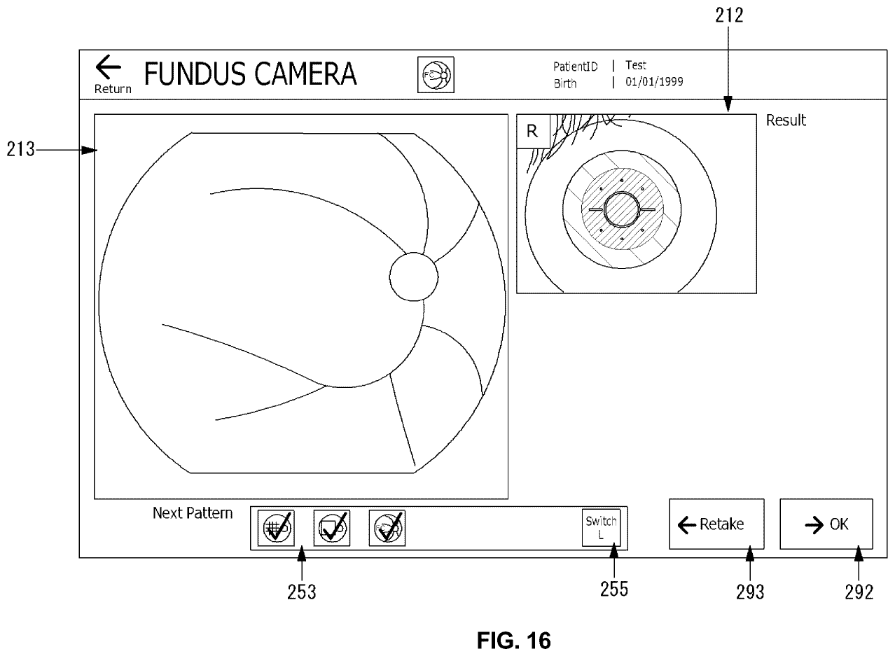

In this case, since no further OCT acquisition is performed, in the observation screen for capturing a fundus front image (color image), no controller or display area relating to OCT is arranged. The remaining points are the same as those in FIGS. 11 and 13. When the Capture button 245 is operated and imaging is performed, the screen shifts to the confirmation screen shown in FIG. 16.

In the confirmation screen shown in FIG. 16, when the OK button 292 is operated, if imaging of the contralateral eye has not yet been performed (S7: No), the imaging unit 3 is moved to the contralateral eye and imaging is repeated for the contralateral eye by the same imaging method. When the various imaging operations corresponding to the selected examination selection button 203 have been completed (S7: Yes), a report output screen (a report list screen in the embodiment) is displayed (S8).

In the present embodiment, the graphic symbols displayed in the progress display box 253 of the confirmation screen also serve as UI elements for changing the next imaging operation to be performed. For example, among the graphic symbols displayed in the progress display area on the confirmation screen, any one of them is arbitrarily selected based on an operation input. After the selection, when the OK button 292 is operated, the process proceeds to the selected imaging operation. Accordingly, a pattern for which imaging is unnecessary in the examination can be skipped as appropriate, and/or the order of the examination can be changed. Further, a left/right eye switching button 255 is arranged in the progress display box 253. When the left/right eye switching button 255 is operated, the process proceeds to an imaging operation for the contralateral eye. Thus, at any stage, the examination can be advanced while switching between the left and right eyes.

### Combo Pattern #3

Next, a description is given of a case in which the button 203c is selected in the OCT/FC mode on the examination selection screen shown in FIG. 4. In this case, an OCT image is captured by a macula line and a macula map. Further, during macula line imaging, axial length data is measured.

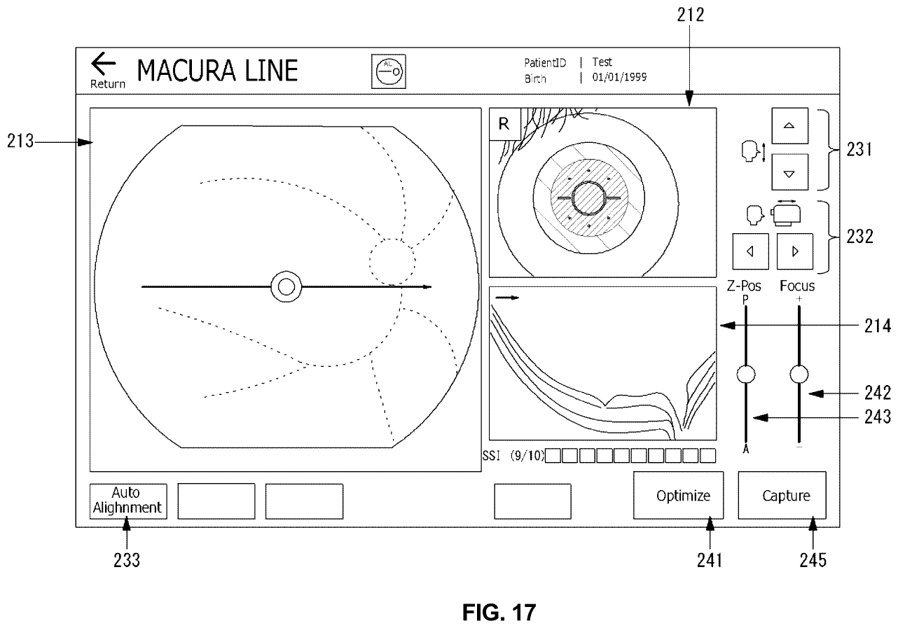

In this case, after completion of alignment, OCT image capturing with the scan pattern of the macula line is first performed. The observation screen displayed during capturing is as shown in FIG. 17. That is, except that the graphic indicating the scan pattern superimposed on the fundus front image 213 is one scan line traversing the macula, the screen configuration and the operation input via the screen are the same as those described for the macula multi.

### Capture and Measurement of Axial Length Data

When the Capture button 245 is operated, a scan for capturing is executed with the scan pattern of the macula line. As described above, when measurement is performed with the scan pattern of the macula line, axial length data is measured. Operation of the Capture button 245 serves as a common trigger for capturing and axial length data measurement. Immediately before the scan for capturing, various types of information for obtaining axial length data of the subject's eye are acquired. Specifically, an interference signal based on a scan for axial length data measurement, an OPL, and Z-direction position information of the imaging unit 3 with respect to the subject's eye E are acquired. Axial length data of the subject's eye E is obtained based on at least the adjusted OPL; in the present embodiment, in order to measure more accurate axial length data, not only the OPL but also an interference signal at the OPL and a Z-direction position of the imaging unit 3 with respect to the subject's eye E are used. That is, from the OPL and the interference signal at the OPL, an optical path length difference corresponding to the position of the fundus can be accurately obtained. In obtaining axial length data, the influence of Z-direction alignment deviation relative to a predetermined working distance position is corrected. Note that an axial length AL of the subject's eye E is represented by the following equation (1), where an axial length of a known model eye is AL0.

AL=AL0+Δp+Δd+Δz (1)

Here, Δp, Δd, and Δz are respectively differences between the above various types of information acquired to obtain an axial length of a subject's eye and reference values based on a known model eye. The reference values may be, for example, the above various types of information acquired in advance at the time of measuring an axial length for the model eye. That is, Δp may be a difference between a measured value and a reference value for the adjusted OPL, Δd may be a difference between a measured value and a reference value for an appearance position of a fundus image based on the interference signal, and Δz may be a difference between a measured value and a reference value for a position in the Z direction of the imaging unit 3 with respect to the subject's eye E.

After completion of the measurement operation of axial length data, a scan for imaging is executed. For example, in the present embodiment, additive imaging may be performed. That is, scanning is performed a plurality of times on the same scan line, and a plurality of B-scan images are captured. In the present embodiment, an interference signal obtained by the scan for imaging is not used for axial length data measurement, and a separate scan is performed for axial length data measurement. Therefore, the measurement operation of axial length data can be executed without depending on a scan pattern of the scan for imaging. That is, implementation of the axial length data measurement function is easy with any imaging scan pattern. Further, axial length data may be measured a plurality of times, and an average value, a median value, or the like may be adopted as a measurement result.

### Display of Confirmation Screen

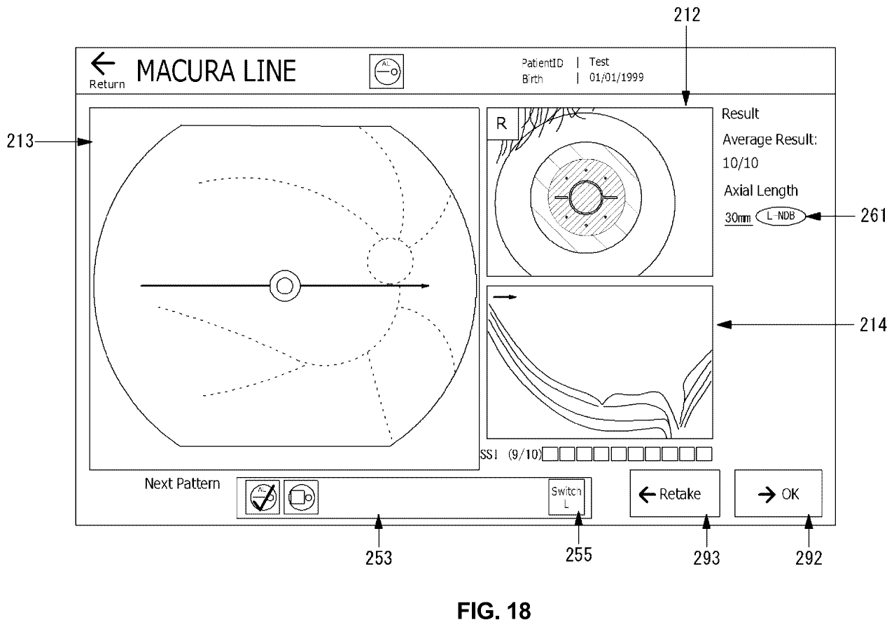

When imaging using the scan pattern of the macular line is completed, the screen shown in FIG. 18 is displayed as a confirmation screen. On this confirmation screen, axial length data are displayed in addition to a tomographic image 214, an anterior-segment observation image 212, and a fundus front image 213. The axial length data are displayed on an axial-length display unit 261. On the axial-length display unit 261, a measurement result of axial length data for the subject's eye E is indicated as a numerical value (e.g., an air-length converted value). When an OK button 292 is operated, the axial length data are stored together with the tomographic image 214. In addition, when the selected combo pattern includes an imaging operation that has not yet been performed, the screen transitions to an observation screen. In this case, the scan pattern or the type of image to be captured is changed to that of the next order.

The graphical symbols displayed in a progress display box 253 of the confirmation screen are also used as UI elements for changing an imaging operation to be performed next, and are operated to appropriately skip a pattern for which imaging is unnecessary in the examination; however, a scan pattern in which measurement of axial length data is performed (in the present embodiment, the macular line) is restricted so as not to be skippable.

On the examination selection screen shown in FIG. 4, when button 203c is selected in the OCT/FC mode, a macular map is captured next. Since the observation screen and the confirmation screen for capturing the macular map would be repetitive, detailed description thereof is omitted.

In OCT thickness analysis obtained from the macular map, a database corresponding to the measured axial length is used. In the present embodiment, the analysis processing is executed immediately after imaging. Therefore, by measuring axial length data before capturing the macular map, analysis is performed after selecting an appropriate database.

As described above, the order of scan patterns for the macular line in which axial length data are measured precedes that of other scan patterns, and skipping imaging using the macular line and changing the order are restricted. Accordingly, the axial length data are reflected in each measurement result and analysis result. Therefore, regardless of how the apparatus is operated in a facility, the accuracy of measurement and analysis for each examination is more likely to be stabilized.

Note that the measurement results and analysis results in which the axial length data are reflected are not limited to the measurement results and analysis results obtained based on other scan patterns included in the combo imaging in which the axial length data were measured. For example, the axial length data may be reflected in measurement results and analysis results for the same subject's eye captured on the same day, or may be reflected in analysis results captured on a later date.

### Report Output Screen (Report List Screen)

When the OK button 292 is operated after imaging is completed with each scan pattern included in the combo pattern, a report output screen is displayed.

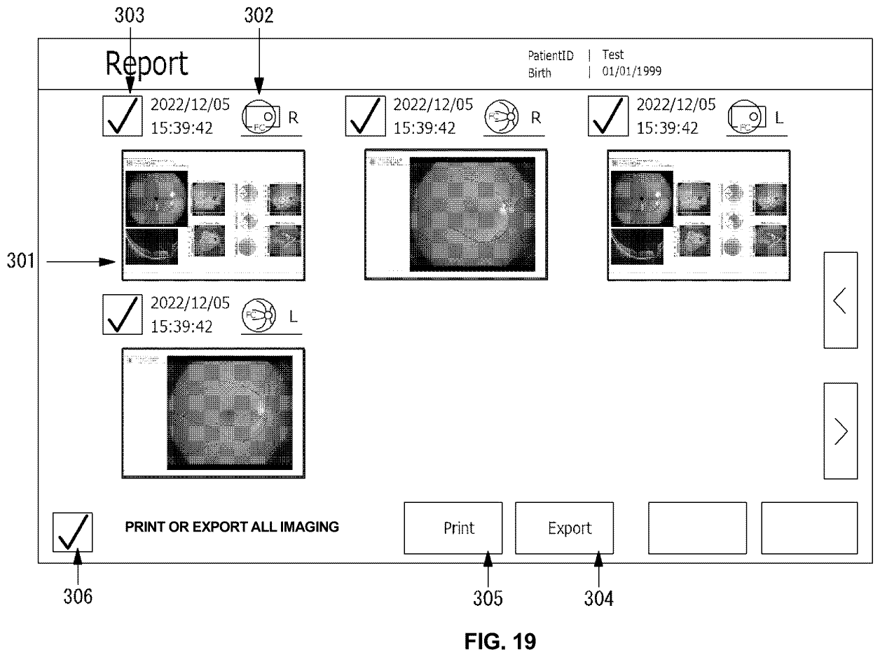

FIG. 19 illustrates an example of the report output screen. In the present embodiment, a report is generated for each imaging operation and for each subject's eye E. When transitioning from the confirmation screen to the report output screen as in the present embodiment, the report output screen displays, side by side, a plurality of reports 301 corresponding to the respective imaging operations in the combo pattern executed immediately before. The plurality of reports 301 displayed side by side on the report output screen are reduced in size. That is, the plurality of reports 301 displayed side by side on the report output screen may be thumbnails. In the report output screen illustrated in FIG. 19, each report may be arranged in the order in which imaging was performed, regardless of the eye to be imaged.

Note that the report output screen exemplified in FIG. 19 is displayed, for convenience of explanation, when, in FIG. 4, the examination selection button 203b indicated by #4 is selected and a continuous pattern, which is a scan pattern in which a wide-area map (retinal map) including the macula and the optic disc and a fundus front image are captured in series, is executed for both eyes.

The report output screen exemplified in FIG. 19 shows a case where two types of reports 301a and 301b are displayed for each of the left and right eyes. The report 301a is a report regarding a wide-area map, and the report 301b is a report regarding a fundus frontal image (here, a color fundus image). In the wide-area map, map scanning is performed with, as an imaging range, a range including both the macula and the optic disc. Further, three-dimensional OCT data are analyzed in each of a first analysis region set around the macula and a second analysis region set around the optic disc. In the report regarding the wide-area map, analysis results (analysis maps and charts) corresponding to the respective analysis regions are displayed. Furthermore, a fundus frontal image and a tomographic image may be displayed in the report regarding the wide-area map.

As shown in FIG. 19, in the report output screen of the present embodiment, around each report 301, a graphic symbol 302 corresponding to an imaging operation on which each report 301a or 301b is based is arranged. Since the graphic symbols arranged around the reports 301a and 301b are the same as the graphic symbols used for the examination selection button 203, which imaging operation each report indicates can be easily understood via the graphic symbols.

In the report output screen, check boxes 303 are arranged in correspondence with the respective reports 301a and 301b. By selecting a check box 303, a check mark is assigned to the report. When the export button 304 or the print button 305 is operated while a check mark has been assigned to any report, the report to which the check mark has been assigned is output. Further, by selecting a check box 303 for collective selection, a check mark can be assigned to all reports or can be cleared.

When the export button 304 is operated, data of the report to which a check mark has been assigned is transferred to a predetermined address. Accordingly, the report is transferred to an external system. The print button 305 causes a printer (not shown) to print out the report to which a check mark has been assigned.

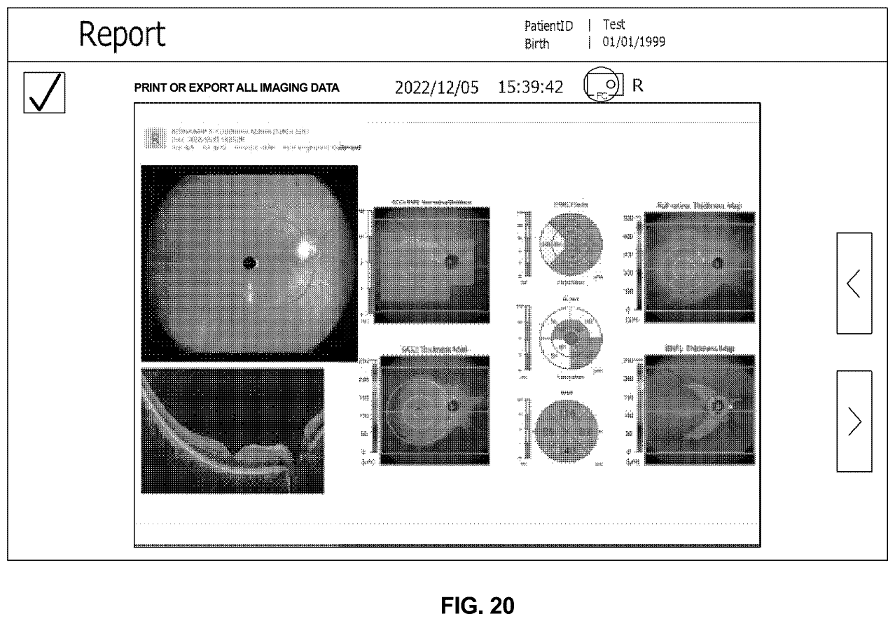

Further, any report selected via the output screen of FIG. 19 can be displayed in an enlarged manner as shown in FIG. 20. Thus, the examiner can check each report in detail.

### Presetting of Combo Patterns

As described above, the combo patterns selectable by operating the examination selection button 203 (see FIGS. 4 to 6) can be set and changed in advance.

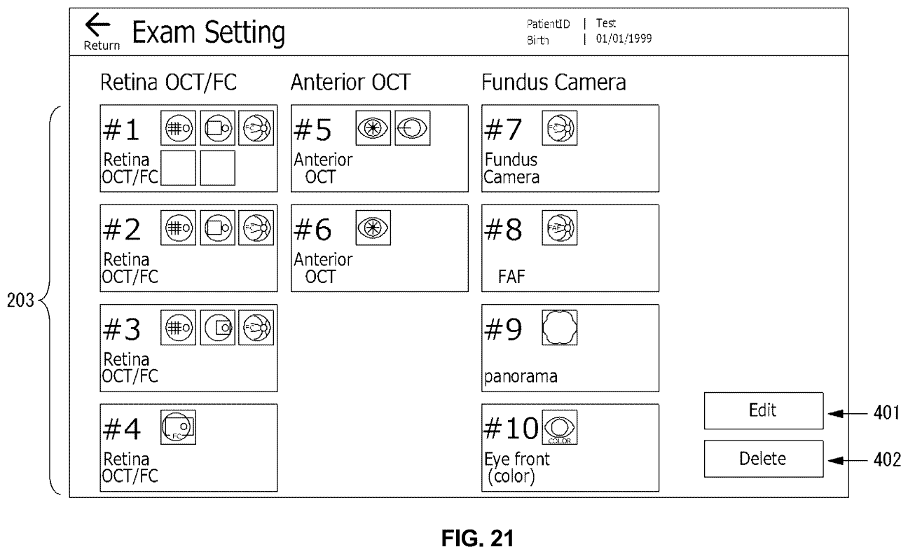

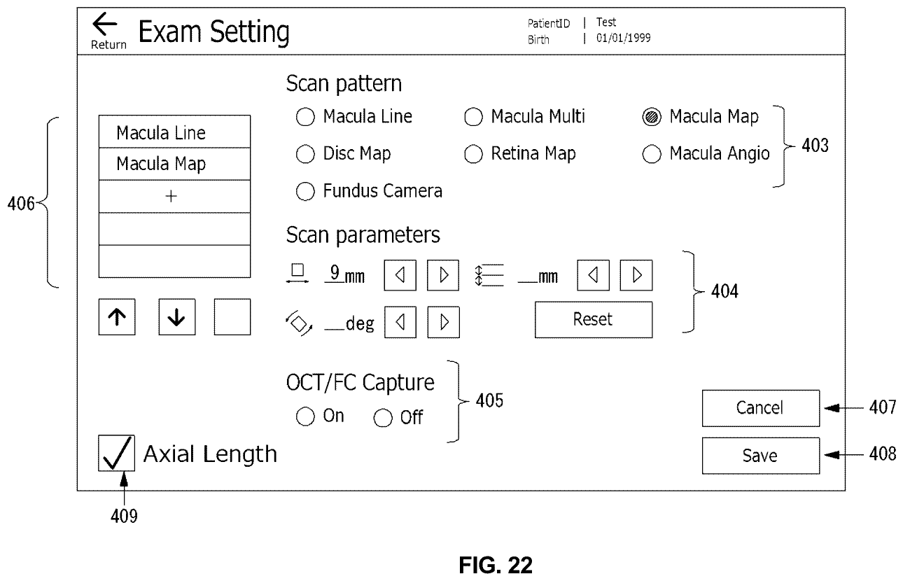

Screens used to set combo patterns are shown in FIGS. 21 and 22. In the screen shown in FIG. 21, examination selection buttons 203 selectable in each mode are displayed in a list.

When any button is selected and the Edit button 401 is operated, the screen transitions to the screen of FIG. 22, and the combo pattern corresponding to the button becomes editable. In the screen of FIG. 22, a combo list 406 is displayed. By selecting a cell on the combo list 406 in which “+” is displayed and selecting any one of a plurality of scan patterns listed in a scan pattern selection unit 403, a scan pattern included in the combo list 406 is added. In the case of a scan pattern for OCT, scan parameters (e.g., scan length, scan pitch, angle, etc.) can be changed from default values via a scan parameter setting unit 404. In the case of a scan pattern for OCT, whether to capture a fundus frontal image in conjunction with OCT imaging is set via an OCT/FC continuous imaging setting unit 405. In an imaging operation in which On is selected, a fundus frontal image (color fundus image) is subsequently captured after OCT imaging without requesting a release operation. That is, a continuous pattern of OCT and a fundus frontal image is set.

Here, a checkbox 409 for incorporating measurement of axial length data into a combo pattern is provided. When a check is entered in the checkbox 409, measurement of axial length data is incorporated among the scan patterns included in the combo list 406.

In the present embodiment, as described above, in the case of a macular line, measurement of axial length data can be incorporated. Among the scan patterns included in the combo list 406, measurement of axial length data can be incorporated only when the scan pattern placed at the top is a macular line. This makes it possible to reliably reflect the axial length data when the axial length data are used to correct or analyze measurement results obtained after the macular line. In this case, the checkbox 409 may be restricted from being turned on when the first scan pattern is a scan pattern in which measurement of axial length data cannot be incorporated. Alternatively, without restricting input to the checkbox 409, when the save button 408 is pressed, it may be determined whether the top scan pattern in the combo list 406 is a scan pattern into which measurement of axial length data can be incorporated (here, whether it is a macular line), and if incorporation is not possible, a warning or the like may be displayed. This makes it possible, for example, even when analysis is performed as needed immediately after imaging of each scan pattern, to obtain appropriate measurement results and analysis results because the axial length has been measured in advance.

Based on the combo list 406 edited via the screen of FIG. 22, a combo pattern corresponding to each examination selection button 203 is set. Also, the pictorial symbol placed on each examination selection button 203 is also changed according to the combo pattern.

### Modified Example

The technique disclosed in the above embodiment is merely an example. Therefore, the technique exemplified in the above embodiment can also be modified. For example, it is also possible to implement only some of the plurality of techniques exemplified in the above embodiment.

### Multiple Settings of Analysis Regions

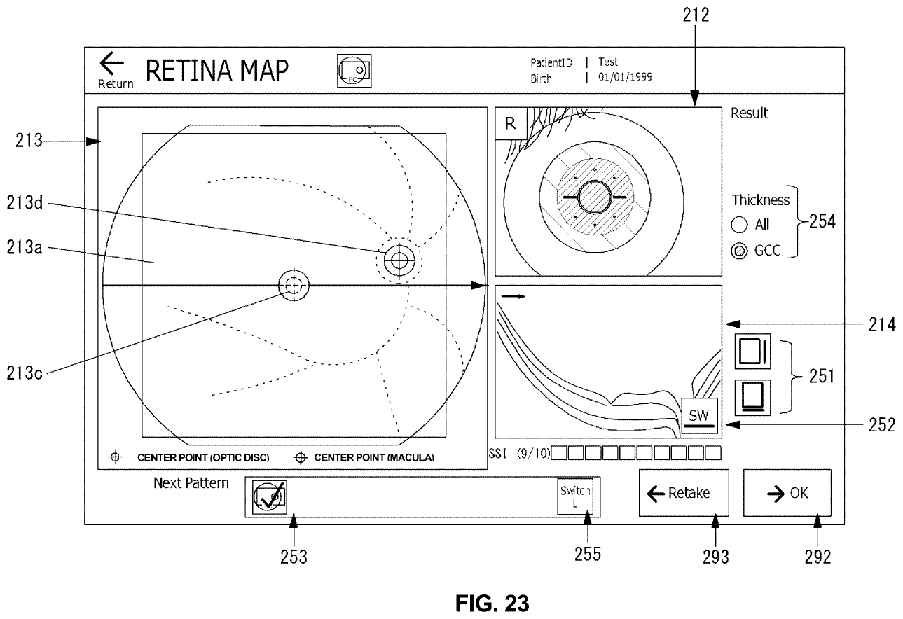

For example, in the above embodiment, when imaging is performed by map scanning, an operation input for changing and setting an analysis region in a three-dimensional tomographic image can be accepted via a confirmation screen. In the above embodiment, one analysis region centered on the macular region is set; however, in some cases, a plurality of analysis regions may be set. For example, when map scanning is performed with an imaging range that includes the macula and the optic disc, three-dimensional OCT data may be analyzed in each of a first analysis region set around the macula and a second analysis region set around the optic disc, thereby generating analysis maps corresponding to the respective analysis regions. In this case, an operation input for changing a reference position for analysis in each of the first analysis region and the second analysis region may be input on the same confirmation screen. For example, as illustrated in FIG. 23, by allowing an operation for moving a first mark 213c indicating a center position of the first analysis region and a second mark 213d indicating a center position of the second analysis region to be input, the reference position of each analysis region may be changeable in the XY directions. An analysis region in a depth direction may also be set individually between the first analysis region and the second analysis region.

### Exemplary Enumeration of Analysis Processing

In the above embodiment, an analysis process regarding tissue thickness has been described as the analysis processing; however, the present disclosure is not necessarily limited thereto, and analysis regarding measured values (actual measured values) based on three-dimensional OCT data other than tissue thickness may be performed, or a result of comparison between the measured values and normal-eye data may be presented. The measured values based on the three-dimensional OCT data may relate to at least one of tissue thickness, density, and shape, etc. For example, the analysis map may be a tissue-thickness map of the eye E (specific examples include a fundus layer-thickness map, a corneal-thickness map, and the like), a density map (specific examples include a vascular-density map, a cell-density map, and the like), a curvature map (topography), a height map (elevation map), or a map indicating a two-dimensional distribution of layer-thickness information or a two-dimensional distribution of vascular information other than those listed here. The normal-eye data may be statistical data in a normal eye with respect to measured values (actual measured values) based on three-dimensional OCT data. The comparison result may be information indicating a degree of deviation between the measured values and the normal-eye data, and may be expressed by, for example, any of a difference, a ratio (e.g., a percentile), and a deviation value. The analysis map may indicate a two-dimensional distribution of temporal changes in the measured values or the above comparison result. In the above embodiment, the three-dimensional OCT data serving as a basis for the analysis map indicates reflection intensity in a three-dimensional region of the eye E (a three-dimensional region obtained by further adding a depth direction to a two-dimensional region in the XY directions); however, the present disclosure is not necessarily limited thereto, and the data may be three-dimensional motion-contrast data.

### Modified Example of Report Output Screen

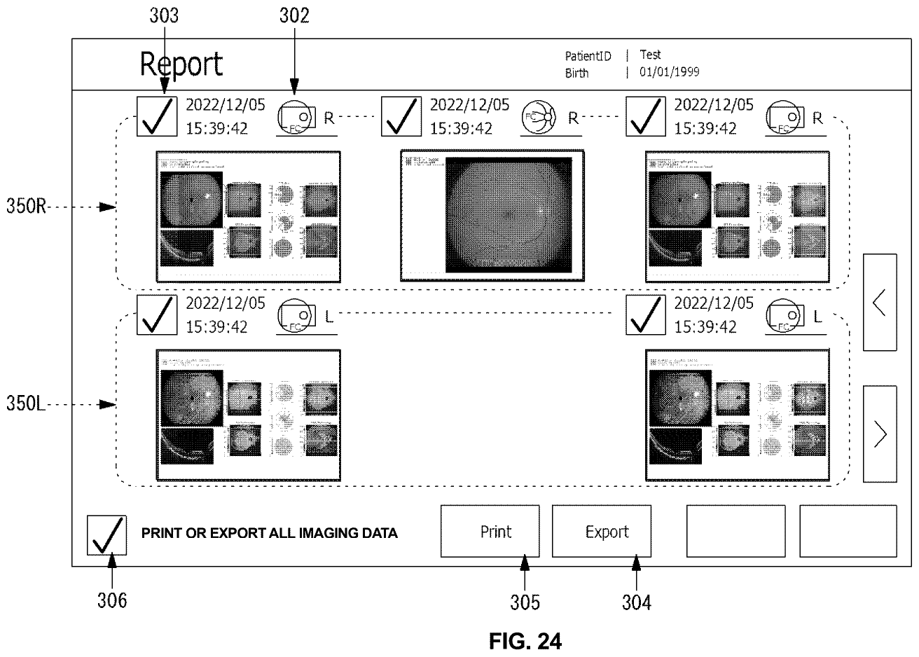

FIG. 24 illustrates a report output screen according to a modified example. In the report output screen of FIG. 24, a right-eye display area 350R and a left-eye display area 350L are set in advance as regions in which reports are arranged. In the example of FIG. 24, the right-eye display area 350R is set to an upper half of the region in which reports are arranged, and the left-eye display area 350L is set to a lower half of the region in which reports are arranged. A report based on an ophthalmic image of the right eye is arranged in the right-eye display area 350R, and a report based on an ophthalmic image of the left eye is arranged in the left-eye display area 350L. Accordingly, an examiner can intuitively grasp the reports respectively corresponding to the right eye and the left eye.

If the same imaging operation is performed on each of the right eye and the left eye, and reports based on the same imaging operation are generated for the right eye and the left eye, those reports of the right eye and the left eye may be arranged at corresponding positions in the right-eye display area 350R and the left-eye display area 350L. As an example, in FIG. 24, since a wide-area map is captured for both the right eye and the left eye, a report regarding the wide-area map is arranged at corresponding positions in the right-eye display area 350R and the left-eye display area 350L (here, vertically). Since a fundus frontal image is captured for the right eye, whereas no fundus frontal image is captured for the left eye, in the right-eye display area 350R, an area of the right-eye display area 350R corresponding to the fundus frontal image is blank. At this time, for each of the right and left eyes, items captured on the same imaging date and with the same scan pattern may be arranged at positions corresponding to each other (here, arranged vertically). In addition, if capturing both eyes is selected by the eye selection button 201 shown in FIGS. 4 to 6, items actually captured with the same scan pattern may be arranged at positions corresponding to each other (here, arranged vertically).

Accordingly, an examiner can intuitively grasp whether ophthalmic images respectively corresponding to the right eye and the left eye have been captured without omission.

The present disclosure can be described as ophthalmic apparatuses A1-A7, B1-B5, and C1-C7 as follows.
(Ophthalmic Apparatus A1) An ophthalmic apparatus A1 includes: a face support unit that supports a subject's face; an examination unit that examines a subject's eye; a drive unit that moves the examination unit three-dimensionally; an imaging unit that acquires an alignment image including at least one of right and left subject's eyes; and a controller, wherein the controller is configured to display the alignment image and display alignment assistance information indicating whether a position of the subject's eye is within a first range on the alignment image that is a movable range of the examination unit by the drive unit or within a second range on the alignment image that is outside the first range.
(Ophthalmic Apparatus A2) An ophthalmic apparatus A2 according to the ophthalmic apparatus A1, wherein, in the ophthalmic apparatus according to the first aspect, the controller visually indicates, as the alignment assistance information, a boundary between the first range and the second range when the alignment image including the first range and the second range is displayed.
(Ophthalmic Apparatus A3) An ophthalmic apparatus A3 according to the ophthalmic apparatus A2, wherein the controller further displays, as the alignment image, an image acquired by performing image processing for reducing visibility in the second range as compared with visibility in the first range.
(Ophthalmic Apparatus A4) An ophthalmic apparatus A4 according to any one of the ophthalmic apparatuses A1-A3, wherein the controller detects the subject's eye from the alignment image and, when the subject's eye is determined to be positioned within the first range on the alignment image, moves the examination unit in accordance with the position of the subject's eye and controls the examination unit to perform an examination on the subject's eye.
(Ophthalmic Apparatus A5) An ophthalmic apparatus A5 according to any one of the ophthalmic apparatuses A1-A4, wherein the controller detects the subject's eye from the alignment image and, when the subject's eye is determined to be positioned within the second range on the alignment image, executes guidance processing for guiding vertical movement of the subject's face.
(Ophthalmic Apparatus A6) An ophthalmic apparatus A6 according to the ophthalmic apparatus A5, further comprising a chin-rest height adjustment mechanism that changes a height of a chin rest included in the face support unit, wherein the controller, in the guidance processing, controls the chin-rest height adjustment mechanism to adjust the height of the chin rest in accordance with a position of the subject's eye on the alignment image.
(Ophthalmic Apparatus A7) An ophthalmic apparatus A7 according to the ophthalmic apparatus A5, further comprising a chin-rest height adjustment mechanism that changes a height of a chin rest included in the face support unit, wherein the controller, in the guidance processing, outputs an operation guide for adjusting the height of the chin rest.
(Ophthalmic Apparatus B1) An ophthalmic apparatus B1 comprises: an imaging unit configured to capture an ophthalmic image of a subject's eye through a plurality of types of imaging operations; a first selection unit configured to accept a first selection operation for selecting a subject; a second selection unit configured to accept a second selection operation for selecting, from among a plurality of pre-prepared examination patterns, an examination pattern including two or more types of imaging operations, the examination pattern indicating examination contents for the selected subject; an imaging control unit configured, when an examination pattern including two or more types of imaging operations is selected and the examination is started, sequentially capture a plurality of ophthalmic images of the subject's eye using the respective imaging operations; and a display control unit configured to generate a plurality of reports based on the plurality of the ophthalmic images acquired by the respective imaging operations for the subject and further display the plurality of reports in a list on a report list screen.
(Ophthalmic Apparatus B2) An ophthalmic apparatus B2 according to the ophthalmic apparatus B1, wherein the report list screen transitions from a confirmation screen or an observation screen.
(Ophthalmic Apparatus B3) An ophthalmic apparatus B3 according to the ophthalmic apparatus B1, wherein, on the report list screen, thumbnails of reports are displayed in a list.
(Ophthalmic Apparatus B4) An ophthalmic apparatus B4 according to any one of the ophthalmic apparatuses B1 to B2, wherein, when an examination pattern for capturing images of each of a right eye and a left eye is selected and the ophthalmic images of each of the right eye and the left eye are captured, the report list screen has a right-eye display area and a left-eye display area that have been preset in advance, the reports based on the ophthalmic images of the right eye are displayed in a list in the right-eye display area, and the reports based on the ophthalmic images of the left eye are displayed in a list in the left-eye display area.
(Ophthalmic Apparatus B5) An ophthalmic apparatus B5 according to the ophthalmic apparatus B4, wherein the reports displayed in the right-eye display area and the reports displayed in the left-eye display area are arranged at positions corresponding to each other in accordance with at least one of a type of an imaging operation on which each report is based and an imaging date.
(Ophthalmic Apparatus C1) An ophthalmic apparatus C1 comprises an examination unit configured to perform a plurality of types of examinations on a subject's eye and a controller configured to control the examination unit, wherein the controller is configured to: execute a combo examination in which two or more of the plurality of types of examinations are successively executed in a predetermined order; accept, during the combo examination, an operation input for skipping any examination whose turn has come; and restrict a specific examination that is one of the plurality of types of examinations from being skipped even when receiving the operation input during the combo examination.
(Ophthalmic Apparatus C2) An ophthalmic apparatus C2 according to the ophthalmic apparatus C1, wherein the controller sets an execution order of the respective examinations during the combo examination such that an order of the specific examination is earlier than the other examinations.
(Ophthalmic Apparatus C3) An ophthalmic apparatus C3 according to the ophthalmic apparatus C1 or C2, wherein the examination unit includes an OCT optical system configured to scan over a tissue of the subject's eye with a plurality of scan patterns, the controller is configured to: successively execute, as the combo examination, two or more of the plurality of scan patterns in a predetermined order; accept an operation input for skipping any scan pattern whose turn has come during the combo examination, and restrict a specific scan pattern that is one of the plurality of types of examinations from being skipped even when receiving the operation input during the combo examination.
(Ophthalmic Apparatus C4) An ophthalmic apparatus C4 according to the ophthalmic apparatus C3, wherein the controller acquires eye axial length information of the subject's eye as an examination result of the subject's eye acquired by the specific scan pattern, and further includes an analysis processing unit that analyzes another scan pattern other than the specific scan pattern, wherein the other scan pattern is not restricted from being skipped when the controller receives the operation input in the combo examination, and the analysis processing unit is configured to perform analysis using at least the axial length.
(Ophthalmic Apparatus C5) An ophthalmic apparatus C5 according to the ophthalmic apparatus C4, wherein the controller restricts a change of an imaging position during an examination of the subject's eye using the specific scan pattern.
(Ophthalmic Apparatus C6) An ophthalmic apparatus C6 according to the ophthalmic apparatus C5, wherein the analysis processing unit corrects a scan length of the other scan patterns based on the axial length acquired as an examination result of the subject's eye using the specific scan pattern.
(Ophthalmic Apparatus C7) An ophthalmic apparatus C7 according to the ophthalmic apparatus C5, wherein the analysis processing unit includes: a first database in which layer thickness information of fundi of a plurality of eyes each having a long axial length with an axial length value exceeding a reference is stored; and a second database in which thickness information of retinal layers of a plurality of subject's eyes each having an axial length with an axial length value within a range of the reference is stored, and the analysis processing unit, in analysis processing using the other scan patterns, refers to at least one of the first database or the second database in accordance with the axial length acquired as an examination result of the subject's eye using the specific scan pattern, and acquires analysis information regarding layer thickness information of the fundus of the subject's eye.

1. An ophthalmic apparatus, comprising:
a face support unit configured to support a subject's face;
an examination unit configured to examine a subject's eye;
a drive unit configured to move the examination unit three-dimensionally;
an imaging unit configured to acquire an alignment image including at least one of left and right subject's eyes; and
a controller configured to display the alignment image, wherein
the controller is further configured to display alignment assistance information indicating whether a position of the subject's eye is within (i) a first range that is a movable range of the examination unit on the alignment image by the drive unit or (ii) a second range on the alignment image that is outside the first range.

2. The ophthalmic apparatus according to claim 1, wherein
when the alignment image including the first range and the second range is displayed, the controller is further configured to visually indicate, as the alignment assistance information, a boundary between the first range and the second range.

3. The ophthalmic apparatus according to claim 2, wherein
the controller is further configured to display, as the alignment image, an image that has been subjected to image processing for reducing visibility in the second range as compared with visibility in the first range.

4. The ophthalmic apparatus according to claim 1, wherein
the controller is further configured to:
detect a position of the subject's eye from the alignment image; and
when the subject's eye is determined to be positioned within the first range on the alignment image, move the examination unit in accordance with the position of the subject's eye and control the examination unit to perform an examination on the subject's eye.

5. The ophthalmic apparatus according to claim 1, wherein
the controller is further configured to:
detect a position of the subject's eye from the alignment image; and
when the subject's eye is determined to be positioned in the second range on the alignment image, perform guidance processing for guiding vertical movement of the subject's face.

6. The ophthalmic apparatus according to claim 5, further comprising
a chin-rest height adjustment mechanism configured to change a height of a chin rest included in the face support unit, wherein
the controller is further configured to, in the guidance processing, control the chin-rest height adjustment mechanism to adjust the height of the chin rest in accordance with the position of the subject's eye on the alignment image.

7. The ophthalmic apparatus according to claim 5, further comprising
a chin-rest height adjustment mechanism configured to change a height of a chin rest included in the face support unit, wherein
the controller is further configured to, in the guidance processing, output an operation guide for adjusting the height of the chin rest.

8. A method, comprising:
supporting, with a face support unit, a subject's face;
examining, with an examination unit, a subject's eye;
moving, with a drive unit, the examination unit three-dimensionally;
acquiring, with an imaging unit, an alignment image including at least one of left and right subject's eyes; and
displaying, with a controller, the alignment image, wherein
the method further comprises displaying alignment assistance information indicating whether a position of the subject's eye is within (i) a first range that is a movable range of the examination unit on the alignment image by the drive unit or (ii) a second range on the alignment image that is outside the first range.

9. The method according to claim 8, further comprising
when the alignment image including the first range and the second range is displayed, visually indicating, with the controller, as the alignment assistance information, a boundary between the first range and the second range.

10. The method according to claim 9, further comprising
displaying, with the controller, as the alignment image, an image that has been subjected to image processing for reducing visibility in the second range as compared with visibility in the first range.

11. The method to claim 8, further comprising:
detecting, with the controller, a position of the subject's eye from the alignment image; and
when the subject's eye is determined to be positioned within the first range on the alignment image, moving, with the controller, the examination unit in accordance with the position of the subject's eye and controlling, with the controller, the examination unit to perform an examination on the subject's eye.

12. The method according to claim 8, further comprising:
detecting, with the controller, a position of the subject's eye from the alignment image; and
when the subject's eye is determined to be positioned in the second range on the alignment image, performing, with the controller, guidance processing for guiding vertical movement of the subject's face.

13. The method according to claim 12, further comprising:
changing, with a chin-rest height adjustment mechanism, a height of a chin rest included in the face support unit; and
in the guidance processing, controlling, with the controller, the chin-rest height adjustment mechanism to adjust the height of the chin rest in accordance with the position of the subject's eye on the alignment image.

14. The method according to claim 12, further comprising:
changing, with a chin-rest height adjustment mechanism, a height of a chin rest included in the face support unit; and
in the guidance processing, outputting, with the controller, an operation guide for adjusting the height of the chin rest.
